(19) United States
(12) Patent Application Publication
(10) Pub. No.: US 2023/0165835 A1
(43) Pub. Date: Jun. 1, 2023

(54) METHODS OF TREATING ACUTE LUNG INJURY USING EBSELEN

(71) Applicant: SOUND PHARMACEUTICALS INC., Seattle, WA (US)

(72) Inventor: Jonathan KIL, Seattle, WA (US)

(21) Appl. No.: 17/921,924

(22) PCT Filed: Apr. 23, 2021
(86) PCT No.: PCT/US2021/028909

§ 371 Date: Oct. 27, 2022

Related U.S. Application Data
(60) Provisional application No. 63/014,822, filed on Apr. 24, 2020.

(51) Int. Cl. A61K 31/41; A61K 45/06; A61K 31/675; A61P 31/14; A61P 11/00

(52) U.S. Cl. CPC A61K 31/41; A61K 45/06; A61K 31/675; A61P 31/14; A61P 11/00

(57) ABSTRACT

Provided herein are methods and compositions related to using ebselen for treating acute lung infections and related conditions or diseases. In certain embodiments the methods relate to treating coronavirus-mediated lung injuries. Also provided are methods of treating a patient who has a confirmed or suspected viral lung infection, comprising administering a therapeutically effective amount of ebselen to a patient suffering from a viral lung infection. Also provided are methods of treating a patient who has or is at risk for cytokine release syndrome (CRS), comprising administering an effective amount of ebselen to a patient who has, or is at risk for, CRS. In another aspect, provided herein is a pharmaceutical comprising ebselen, and an antiviral agent that is useful in the methods of this disclosure.

### 1. CROSS-REFERENCE TO RELATED APPLICATIONS

The present application claims the benefit of U.S. Provisional Patent Application No. 63/014,822, filed on Apr. 24, 2020, which is hereby incorporated by reference in its entirety.

### 2. INTRODUCTION

There is a critical need for therapies to manage symptoms associated with acute lung injury (ALI), in particular ALI caused by viral respiratory infections, including ALI caused by infection with coronavirus, notably infection with SARS-CoV-2. Patients with acute lung injury are at risk of progressing to intubation for ventilatory support, with a particularly difficult and unpredictable treatment course made especially bleak by the potential for rationing of mechanical ventilators and intensive care unit admission and support. ALI is associated with cellular infiltration of the airways and inflammation. High mobility group box protein 1 (HMGB1) and interleukin-6 (IL-6) are among the pro-inflammatory cytokines implicated in ALI. While agents that specifically inhibit IL-6, such as tocilizumab, and agents that are more generally anti-inflammatory, such as hydroxychloroquine, are currently in clinical trials in patients with COVID-19 with ALI, there presently are no proven effective treatments for ALI, including that caused by coronavirus infections, especially agents that prevent the progression to intubation for ventilatory support.

Jin et al. (Structure of M^{pro} from COVID-19 virus and discovery of its inhibitors. Nature https://doi.org/10.1038/s41586-020-2223-y (2019)) describe studies elucidating the structure of the COVID-19 virus main protease (MP^{pro}) which mediates viral replication and transcription. Jin et al. used virtual screening and high throughput screening methods to identify a number of compounds that could bind to the M^{pro} protease. Several compounds, including ebselen, showed modest in vitro inhibition activity against M^{pro} protease. Further studies are needed to identify and develop an antiviral agent that would be suitable for clinical use against CoV-associated diseases.

Ebselen is an anti-inflammatory compound with glutathione peroxidase (GPx1) activity. GPx1 is the dominant catalytic antioxidant enzyme in the mammalian inner ear, and its activity is decreased after noise or ototoxic insult. Several preclinical and clinical studies have demonstrated the safety and efficacy of SPI-1005 (ebselen) at preventing and treating different forms of sensorineural hearing loss. SPI-1005 capsules, containing 200 mg ebselen, have demonstrated the potential for a safe, well tolerated, oral treatment for Meniere's Disease, a disease for which there are no FDA-approved treatments. Ebselen treatment has been shown to prevent or reverse the pathologic changes in the cochlea following noise- or ototoxin-induced injury, resulting in improved physiology measured by several types of auditory stimuli.

The timely development of effective antiviral agents demonstrated for clinical use is extremely challenging. In view of the high morbidity and mortality that can follow ALI, there is an urgent need for new clinical treatments in patients with COVID-19 with ALI.

### 3. SUMMARY

Disclosed herein are methods of treating a patient who has or is at risk for acute lung injury (ALI), acute respiratory distress syndrome (ARDS), ALI with concomitant pneumonia, or ARDS with concomitant pneumonia, comprising: administering an effective amount of ebselen to a patient who has or is at risk for acute lung injury (ALI), acute respiratory distress syndrome (ARDS), ALI with concomitant pneumonia, or ARDS with concomitant pneumonia.

Additionally, disclosed herein are methods of treating a patient who has a confirmed or suspected viral lung infection, comprising: administering a therapeutically effective amount of ebselen to a patient suffering from a viral lung infection.

Additionally, disclosed herein are methods of treating a patient who has or is at risk for cytokine release syndrome (CRS), comprising: administering an effective amount of ebselen to a patient who has, or is at risk for, CRS.

In another aspect, provided herein is a pharmaceutical comprising ebselen, and an antiviral agent.

### 4. BRIEF DESCRIPTION OF THE DRAWINGS

These and other features, aspects, and advantages of the present invention will become better understood with regard to the following description, and accompanying drawings, where:

FIGS. 1A-C show that ebselen inhibited viral load in an in vitro kidney cell assay (Vero E6 cells), with comparison to remdesivir. Further details are provided in section 6.7

FIG. 2 shows that treatment with ebselen caused an improvement in lung functioning (FEV1) at both 2 and 4 weeks in cystic fibrosis patients experiencing ototoxicity induced by tobramycin. This improvement was higher than what was predicted from an observation study done with patients that were treated with tobramycin only. Further details are provided in section 6.9.1.

### 5. DETAILED DESCRIPTION

Viral infection of the lungs, and the subsequent inflammatory response to clear the invading viral pathogens, including cytokine release syndrome (CRS, colloquially known as “cytokine storm”), significantly contributes to the morbidity and mortality of patients suffering from ALI. ALI can also occur independently of viral infection.

Ebselen (also called SPI-1005; CAS No. 60940-34-3; 2-phenyl-1,2-benzisoselenazol-3(2H)-one) is an anti-inflammatory compound with glutathione peroxidase (GPx1) activity.

Ebselen has been demonstrated as safe in various clinical studies. We have studied the efficacy of ebselen in a number of indications including the treatment of Meniere's Disease, and in the treatment of ototoxicity in cystic fibrosis (CF) patients. Ebselen has an anti-inflammatory mechanism of action that is distinct from that of a classic corticosteroid immunosuppressive agent which can be beneficial in the treatment of respiratory viral infections. In the course of the above clinical studies, we performed several minimum bactericidal concentration (MIC) assays against 18 common microbes to show a lack of interference to support testing in CF patients. In addition, our Phase 2b study in cystic fibrosis (CF) patients with Pseudomonas is continuing without any safety issues. Furthermore, in another clinical study, we have demonstrated improvement in auditory function in Meniere's Disease (hearing loss, word recognition and tinnitus) that exceeds reported improvements with corticosteroid treatment in this population. Our results and analysis in these other clinical studies supports the use of ebselen in the subject methods of treating or preventing conditions associated with respiratory viral infections described herein.

As described herein, provided are methods for treating or preventing ALI, CRS, and/or respiratory viral infections by administering an effective amount of ebselen.

### 5.1. Definitions

Unless defined otherwise, all technical and scientific terms used herein have the meaning commonly understood by one of ordinary skill in the art to which the invention pertains.

As used herein, the terms “patient” and “subject” are used interchangeably, and may be taken to mean any living organism which may be treated with compounds of the present invention. As such, the terms “patient” and “subject” include, but are not limited to, any non-human mammal, primate and human.

A “therapeutically effective amount” of a composition is an amount sufficient to achieve a desired therapeutic effect, and therefore does not require cure or complete remission.

The terms “treat,” “treated,” “treating”, or “treatment” as used herein have the meanings commonly understood in the medical arts, and therefore do not require cure or complete remission, and therefore include any beneficial or desired clinical results. Nonlimiting examples of such beneficial or desired clinical results are prolonging survival as compared to expected survival if not receiving treatment, reduced probability of requiring intubation and mechanical ventilation, reduced number of days on mechanical ventilation, reduced days in the ICU, reduced days of total hospitalization.

As used herein “preventing” a disease refers to inhibiting the full development of a disease.

As used herein, “interleukin 6 (IL-6)” or “IL-6 polypeptide” refers to a human polypeptide or fragment thereof having at least about 85% or greater amino acid identity to the amino acid sequence provided at NCBI Accession No. NP 000591 and having IL-6 biological activity. IL-6 is a pleotropic cytokine with multiple biologic functions. Exemplary IL-6 biological activities include immunostimulatory and pro-inflammatory activities.

Unless otherwise specified, “IL-6 antagonist” is used synonymously with “IL-6 inhibitor” and refers to an agent that is capable of decreasing the biological activity of IL-6. IL-6 antagonists include agents that decrease the level of IL-6 polypeptide in serum, including agents that decrease the expression of an IL-6 polypeptide or nucleic acid; agents that decrease the ability of IL-6 to bind to the IL-6R; agents that decrease the expression of the IL-6R; and agents that decrease signal transduction by the IL-6R receptor when bound by IL-6. In preferred embodiments, the IL-6 antagonist decreases IL-6 biological activity by at least about 10%, 20%, 30%, 50%, 70%, 80%, 90%, 95%, or even 100%. As further described below, IL-6 antagonists include IL-6 binding polypeptides, such as anti-IL-6 antibodies and antigen binding fragments or derivatives thereof; IL-6R binding polypeptides, such as anti-IL-6R antibodies and antigen binding fragments or derivatives thereof; and synthetic chemical molecules, such as JAK1 and JAK3 inhibitors.

The term “IL-6 antibody” or “anti-IL-6 antibody” refers to an antibody that specifically binds IL-6 ligand. Anti-IL-6 antibodies include monoclonal and polyclonal antibodies that are specific for IL-6 ligand, and antigen-binding fragments or derivatives thereof. IL-6 antibodies are described in greater detail below.

The term “C-reactive protein” or “CRP” refers to a polypeptide or fragment thereof having at least about 85% or greater amino acid identity to the amino acid sequence provided at NCBI Accession No. NP 000558 and having complement activating activity. CRP levels increase in response to inflammation, and can be measured with an hsCRP (high-sensitivity C-reactive protein) test.

The term “biological sample” refers to any tissue, cell, fluid, or other material derived from an organism (e.g., human subject). In certain embodiments, the biological sample is serum or blood.

As used herein, “pre-treatment” means prior to the first administration of ebselen according the methods described herein. Pre-treatment does not exclude, and often includes, the prior administration of treatments other than ebselen.

As used herein, “post-treatment” means after the administration of ebselen according the methods described herein. Post-treatment includes after any administration of ebselen at any dosage described herein. Post-treatment also includes after the bolus treatment phase of ebselen, and also after continuous administration of ebselen at any dosage described herein.

Any convenient biological indicators, e.g., as described herein can be utilized as biomarkers indicating a patient in need for treatment with ebselen. Additionally, any one of combination of these biomarkers can be utilized to show an improvement in patient outcome by exhibiting an improved level following treatment with ebselen.

### 5.2. Methods of Treating Virus-Mediated Lung Injury and Related Conditions

In an aspect, the present disclosure provides a method of treating a patient who has or is at risk for acute lung injury (ALI), acute respiratory distress syndrome (ARDS), ALI with concomitant pneumonia, or ARDS with concomitant pneumonia. The method comprises administering an effective amount of ebselen to a patient who has or is at risk for acute lung injury (ALI), acute respiratory distress syndrome (ARDS), ALI with concomitant pneumonia, or ARDS with concomitant pneumonia.

In an aspect, the present disclosure provides a method of treating a patient who has a confirmed or suspected viral lung infection. The method comprises administering a therapeutically effective amount of ebselen to a patient suffering from a viral lung infection. In certain embodiments, the patient has or is at risk for ALI. In certain embodiments, the patient has or is at risk of ARDS. In certain embodiments, the patient has or is at risk for ALI with concomitant pneumonia or ARDS with concomitant pneumonia.

In an aspect, the present disclosure provides a method of treating a patient who has or is at risk for cytokine release syndrome (CRS), the method comprising administering an effective amount of ebselen to a patient who has or is at risk for CRS. In various embodiments, the patient who has or is at risk for CRS is determined to have or at risk for ALI, ARDS, ALI with concomitant pneumonia, or ARDS with concomitant pneumonia. In various embodiments, the patient who has or is at risk for CRS has confirmed or suspected viral lung infection.

While not wishing to be bound by theory, the present disclosure is based at least in part on the ability of an effective amount of ebselen to alleviate, diminish or prevent one or more of the symptoms associated with these conditions, including CRS, as described herein below.

5.2.1. Pre-Treatment Symptoms and Signs

5.2.1.1 Confirmed or Suspected Viral Lung Infection

In various embodiments of the methods described herein, the patient has a confirmed or suspected viral lung infection.

In some embodiments, the infection is by a virus selected from coronavirus, influenza virus, rhinovirus, respiratory syncytial virus, metapneumovirus, adenovirus, and boca virus.

In some embodiments, the virus is a coronavirus. In certain embodiments, the virus is any one or combination of the following coronaviruses: coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19). In a specific embodiment, the virus is SARS-CoV-2, also referred to as nCoV-2, nCoV2 or 2019-nCoV. The terms “nCoV2”, “nCoV-2” and “SARS-CoV-2” are used interchangeably herein. In particular embodiments, the patient has severe acute respiratory syndrome (SARS). In particular embodiments, the patient has middle eastern respiratory syndrome (MERS). In particular embodiments, the patient has coronavirus disease 2019 (COVID-19).

In some embodiments, the virus is an influenza virus. In particular embodiments, the virus is any one or combination of the following influenza viruses: parainfluenza virus 1, parainfluenza virus 2, parainfluenza virus 3, parainfluenza virus 4, influenza A virus, and influenza B virus.

In various embodiments, viral infection has been or is concomitantly confirmed by detection of viral genetic material in a fluid sample from the patient. In some embodiments, viral infection has not been or is not concomitantly confirmed by detection of viral genetic material in a fluid sample from the patient, but is suspected based on clinical presentation and history. In particular embodiments, treatment is initiated before confirmation by detection of viral genetic material. In specific embodiments, treatment is initiated before confirmation by detection of viral genetic material, and viral infection is later confirmed by detection of viral genetic material or virus-specific IgM and/or IgG in the patient's serum.

5.2.1.2 Fever

In some embodiments, the patient has fever. In some embodiments, the patient has a body temperature greater than 37.5° C. In some embodiments, the body temperature is 37.6° C. or greater, 37.7° C. or greater, 37.8° C. or greater, 37.9° C. or greater, 38° C. or greater, 38.1° C. or greater, 38.2° C. or greater, 38.3° C. or greater, 38.4° C. or greater, 38.5° C. or greater, 38.6° C. or greater, 38.7° C. or greater, 38.8° C. or greater, 38.9° C. or greater, 39° C. or greater, 39.1° C. or greater, 39.2° C. or greater, 39.3° C. or greater, 39.4° C. or greater, 39.5° C. or greater, 39.6° C. or greater, 39.7° C. or greater, 39.8° C. or greater, 39.9° C. or greater, 40° C. or greater, 40.1° C. or greater, 40.2° C. or greater, 40.3° C. or greater, 40.4° C. or greater, 40.5° C. or greater, 40.6° C. or greater, 40.7° C. or greater, 40.8° C. or greater, 40.9° C. or greater, 41° C. or greater, or 42° C. or greater. In some embodiments, the patient has a body temperature greater than 37.5° C. for 24 hours or more, 48 hours or more, 72 hours or more, 96 hours or more, 5 days or more, 6 days or more, 1 week or more, 1.5 weeks or more, or 2 weeks or more. In typical embodiments, the body temperature is measured from clinically accessible measurement sites on the patient. In various embodiments, the measurement site is the patient's forehead, temple, and/or other external body surfaces. In some embodiments, the measurement site is the oral cavity, rectal cavity, axilla area, or tympanic membrane.

5.2.1.3 Reduced Blood Oxygen Saturation

In some embodiments, the patient has a blood oxygen saturation level (SpO₂) of less than 95%. In some embodiments, the patient has a blood oxygen saturation level (SpO₂) of less than 94%. In some embodiments, the patient has a blood oxygen saturation level (SpO₂) of 93% or less. In some embodiments, the patient has an SpO₂ level of 92% or less, 91% or less, 90% or less, 85% or less, 80% or less, 75% or less, 70% or less, 65% or less, 60% or less, 55% or less, 50% or less, 45% or less, 40% or less, 35% or less, 30% or less, or 25% or less. In some embodiments, the patient requires mechanical ventilation and/or supplemental oxygen.

5.2.1.4 Pneumonia

In some embodiments, the patient has pneumonia.

5.2.1.5 Hospitalization

In some embodiments, the patient is hospitalized.

5.2.1.6 Mechanical or Assisted Ventilation

In some embodiments, the patient is on a ventilator. In some embodiments, the patient is not on a ventilator.

5.2.1.7 Pre-Treatment d-Dimer and Sepsis-Induced Coagulopathy (SIC) Score

In certain embodiments, the patient has elevated pre-treatment levels of d-dimer above baseline (e.g. >1 μg/ml, or elevated at or above the upper limit of normal). In certain embodiments, the patient has elevated pre-treatment levels of sepsis-induced coagulopathy (SIC) total score of 4 or more with total score of prothrombin time and coagulation exceeding 2, or a score at or above the upper limit of normal.

5.2.1.8 Pre-Treatment Serum CRP and IL-6 Levels

In some embodiments, the patient has elevated pre-treatment levels of serum C-Reactive Protein (CRP).

In some embodiments, the patient has a pre-treatment CRP level of at least 2 mg/L. In some embodiments, the patient has a pre-treatment CRP level of at least 5 mg/L. In some embodiments, the patient's pre-treatment CRP level is at least 2 mg/L, 2.5 mg/L, 3 mg/L, 3.5 mg/L, 4 mg/L, 4.5 mg/L, or 5 mg/L. In some embodiments, the patient has pre-treatment CRP levels of at least 7.5 mg/L, 10 mg/L, 12.5 mg/L, or 15 mg/L. In certain embodiments, the patient's pre-treatment CRP level is at least 7.5 mg/L. In certain embodiments, the patient has a pre-treatment CRP level of at least 10 mg/L. In certain embodiments, the patient has a pre-treatment CRP level of at least 12.5 mg/L. In certain embodiments, the patient has a pre-treatment CRP level of at least 15 mg/L. In certain preferred embodiments, the patient has a pre-treatment CRP level of at least 10 mg/L. In some embodiments, the patient has pre-treatment CRP levels of at least 20 mg/L, 25 mg/L, 30 mg/L, 35 mg/L, 40 mg/L, 45 mg/L, or 50 mg/L. In certain embodiments, the patient's pre-treatment CRP level is at least 20 mg/L. In certain embodiments, the patient has a pre-treatment CRP level of at least 25 mg/L. In certain embodiments, the patient has a pre-treatment CRP level of at least 30 mg/L. In certain embodiments, the patient has a pre-treatment CRP level of at least 35 mg/L. In certain embodiments, the patient's pre-treatment CRP level is at least 40 mg/L. In certain embodiments, the patient has a pre-treatment CRP level of at least 45 mg/L. In certain embodiments, the patient has a pre-treatment CRP level of at least 50 mg/L. In certain preferred embodiments, the patient has a pre-treatment CRP level of at least 40 mg/L.

In some embodiments of the methods described herein, the patient has elevated pre-treatment serum levels of IL-6. In some embodiments, the patient has a pre-treatment serum IL-6 level of at least 2 pg/ml. In various embodiments, the patient has a pre-treatment serum IL-6 level of at least 2 pg/ml, at least 3 pg/ml, at least 4 pg/ml, at least 5 pg/ml, at least 6 pg/ml, at least 7 pg/ml, at least 8 pg/ml, at least 9 pg/ml, at least 10 pg/ml, at least 11 pg/ml, at least 12 pg/ml, at least 13 pg/ml, at least 14 pg/ml, or at least 15 pg/ml. In certain embodiments, the patient has a pre-treatment serum IL-6 level of at least 2.5 pg/ml. In certain embodiments, the patient has a pre-treatment serum IL-6 level of at least 4 pg/ml. In certain embodiments, the patient has a pre-treatment serum IL-6 level of at least 5 pg/ml. In certain embodiments, the patient has a pre-treatment serum IL-6 level of at least 7.5 pg/ml. In certain embodiments, the patient has a pre-treatment serum IL-6 level of at least 10 pg/ml. In certain embodiments, the patient has a pre-treatment serum IL-6 level of at least 12.5 pg/ml. In certain embodiments, the patient has a pre-treatment serum IL-6 level of at least 15 pg/ml. In some embodiments, the patient has a pre-treatment serum IL-6 level of at least 20 pg/ml. In various embodiments, the patient has a pre-treatment serum IL-6 level of at least 20 pg/ml, at least 30 pg/ml, at least 40 pg/ml, at least 50 pg/ml, at least 60 pg/ml, at least 70 pg/ml, at least 80 pg/ml, at least 90 pg/ml, at least 100 pg/ml, at least 150 pg/ml, or at least 200 pg/ml. In certain embodiments, the patient has a pre-treatment serum IL-6 level of at least 30 pg/ml. In certain embodiments, the patient has a pre-treatment serum IL-6 level of at least 40 pg/ml. In certain embodiments, the patient has a pre-treatment serum IL-6 level of at least 50 pg/ml. In certain embodiments, the patient has a pre-treatment serum IL-6 level of at least 75 pg/ml. In certain embodiments, the patient has a pre-treatment serum IL-6 level of at least 100 pg/ml. In certain embodiments, the patient has a pre-treatment serum IL-6 level of at least 150 pg/ml. In certain embodiments, the patient has a pre-treatment serum IL-6 level of at least 200 pg/ml.

In some embodiments, the patient has elevated pre-treatment serum levels of CRP and elevated pre-treatment IL-6 levels. In certain embodiments, the patient has a pre-treatment serum IL-6 level of at least 2 pg/ml and a pre-treatment CRP level of at least 2 mg/L. In certain embodiments, the patient has a pre-treatment serum IL-6 level of at least 2 pg/ml and a pre-treatment CRP level of at least 2.5 mg/L. In certain embodiments, the patient has a pre-treatment serum IL-6 level of at least 2 pg/ml and a pre-treatment CRP level of at least 5 mg/L. In certain embodiments, the patient has a pre-treatment serum IL-6 level of at least 2 pg/ml and a pre-treatment CRP level of at least 10 mg/L. In certain embodiments, the patient has a pre-treatment serum IL-6 level of at least 4 pg/ml and a pre-treatment CRP level of at least 2 mg/L. In certain embodiments, the patient has a pre-treatment serum IL-6 level of at least 4 pg/ml and a pre-treatment CRP level of at least 2.5 mg/L. In certain embodiments, the patient has a pre-treatment serum IL-6 level of at least 4 pg/ml and a pre-treatment CRP level of at least 5 mg/L. In certain embodiments, the patient has a pre-treatment serum IL-6 level of at least 4 pg/ml and a pre-treatment CRP level of at least 10 mg/L. In certain embodiments, the patient has a pre-treatment serum IL-6 level of at least 5 pg/ml and a pre-treatment CRP level of at least 2 mg/L. In certain embodiments, the patient has a pre-treatment serum IL-6 level of at least 5 pg/ml and a pre-treatment CRP level of at least 2.5 mg/L. In certain embodiments, the patient has a pre-treatment serum IL-6 level of at least 5 pg/ml and a pre-treatment CRP level of at least 5 mg/L. In certain embodiments, the patient has a pre-treatment serum IL-6 level of at least 5 pg/ml and a pre-treatment CRP level of at least 10 mg/L. In certain embodiments, the patient has a pre-treatment serum IL-6 level of at least 10 pg/ml and a pre-treatment CRP level of at least 2 mg/L. In certain embodiments, the patient has a pre-treatment serum IL-6 level of at least 10 pg/ml and a pre-treatment CRP level of at least 2.5 mg/L. In certain embodiments, the patient has a pre-treatment serum IL-6 level of at least 10 pg/ml and a pre-treatment CRP level of at least 5 mg/L. In certain embodiments, the patient has a pre-treatment serum IL-6 level of at least 10 pg/ml and a pre-treatment CRP level of at least 10 mg/L.

In some embodiments, the patient has a pre-treatment serum IL-6 level of at least 10 pg/ml and a pre-treatment CRP level of at least 10 mg/L. In certain embodiments, the patient has a pre-treatment serum IL-6 level of at least 10 pg/ml and a pre-treatment CRP level of at least 20 mg/L. In certain embodiments, the patient has a pre-treatment serum IL-6 level of at least 10 pg/ml and a pre-treatment CRP level of at least 30 mg/L. In certain embodiments, the patient has a pre-treatment serum IL-6 level of at least 10 pg/ml and a pre-treatment CRP level of at least 40 mg/L. In certain embodiments, the patient has a pre-treatment serum IL-6 level of at least 20 pg/ml and a pre-treatment CRP level of at least 10 mg/L. In certain embodiments, the patient has a pre-treatment serum IL-6 level of at least 20 pg/ml and a pre-treatment CRP level of at least 20 mg/L. In certain embodiments, the patient has a pre-treatment serum IL-6 level of at least 20 pg/ml and a pre-treatment CRP level of at least 30 mg/L. In certain embodiments, the patient has a pre-treatment serum IL-6 level of at least 20 pg/ml and a pre-treatment CRP level of at least 40 mg/L. In certain embodiments, the patient has a pre-treatment serum IL-6 level of at least 30 pg/ml and a pre-treatment CRP level of at least 10 mg/L. In certain embodiments, the patient has a pre-treatment serum IL-6 level of at least 30 pg/ml and a pre-treatment CRP level of at least 20 mg/L. In certain embodiments, the patient has a pre-treatment serum IL-6 level of at least 30 pg/ml and a pre-treatment CRP level of at least 30 mg/L. In certain embodiments, the patient has a pre-treatment serum IL-6 level of at least 30 pg/ml and a pre-treatment CRP level of at least 40 mg/L. In certain embodiments, the patient has a pre-treatment serum IL-6 level of at least 40 pg/ml and a pre-treatment CRP level of at least 10 mg/L. In certain embodiments, the patient has a pre-treatment serum IL-6 level of at least 40 pg/ml and a pre-treatment CRP level of at least 20 mg/L. In certain embodiments, the patient has a pre-treatment serum IL-6 level of at least 40 pg/ml and a pre-treatment CRP level of at least 30 mg/L. In certain embodiments, the patient has a pre-treatment serum IL-6 level of at least 40 pg/ml and a pre-treatment CRP level of at least 40 mg/L.

5.2.1.9 Pre-Treatment Neutrophil-to-Lymphocyte Ratio

In some embodiments, the patient has a pre-treatment neutrophil-to-lymphocyte ratio (NLR) greater than 2.0. In some embodiments, the patient has a pre-treatment NLR greater than 3.0. In some embodiments, the patient has a pre-treatment NLR greater than 2.8, 2.9, 3.0, 3.1, 3.2, 3.3, 3.4, 3.5, 3.6, 3.7, 3.8, or 3.9. In some embodiment, the patient has a pre-treatment NLR greater than 4.0.

5.2.1.10 Patient Age

In some embodiments, the patient is older than 60 years old. In some embodiments, the patient is older than 50 years old. In some embodiments, the patient is older than 50, 51, 52, 53, 54, 55, 56, 57, 58, 59, or 60 years old. In some embodiments, the patient is younger than 60, 59, 58, 57, 56, 55, 54, 53, 52, 51, or 50 years old. In some embodiments the patient is a young adult between the age of 20-35. In some embodiments, the patient is middle aged, between the age of 35-50. In some embodiments, the patient is a teenager between the age of 13-19. In some embodiments, the patient is a child between the age of 5-12. In alternative embodiments, the patient is a toddler between the age of 1-4. In further embodiments, the patient is an infant between the age of newborn to one year old.

5.2.2. Ebselen

In the methods described herein, the patient is administered an effective amount of ebselen.

5.2.2.1 Drug Substance

Ebselen is a redox-active synthetic organic selenium compound that is able to modify cysteine residues in proteins and enzymes effectively and selectively. In the presence of elevated levels of reactive oxygen species, ebselen can have glutathione peroxidase (GPx)-like activity. Ebselen can be prepared by a variety of methods and is commercially available. Ebselen is slightly soluble in aqueous solutions at 25° Celsius. An embodiment of an ebselen formulation is >99% pure as confirmed by HPLC. Some of the crystalline forms for the ebselen compound may exist as polymorphs and as such are intended to be included in the present disclosure. In addition, the ebselen compound may form solvates with water (i.e., hydrates) or common organic solvents, and such solvates are intended to be encompassed by some embodiments.

5.2.2.2 Pharmaceutical Compositions

In typical embodiments of the methods described herein, ebselen is administered as or is diluted from a pharmaceutical composition. The pharmaceutical composition comprises ebselen and at least one diluent or excipient. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, 8th revised ed. (2017), incorporated herein by reference in its entirety.

Ebselen can be formulated in any appropriate pharmaceutical composition for administration by any suitable route of administration. Suitable routes of administration include, but are not limited to, oral, intravenous, subcutaneous, pulmonary (including pulmonary administration by oral inhalation), and intranasal. A particularly preferred route of administration for use in the methods described herein is oral administration. In some embodiments, ebselen is administered via nasogastric (ng) tube, where the patient is one who is intubated or unable to receive a preferred route of administration.

In some embodiments, the ebselen is formulated for oral administration in a solid dosage form. In certain embodiments, the solid dosage form is a tablet. In certain embodiments, the solid dosage form is a capsule.

In some embodiments, ebselen is the only active ingredient administered in a formulation.

5.2.3. Ebselen Dosage Regimens

In various embodiments of the methods described herein, ebselen is administered by a route selected from oral, intravenous, subcutaneous, pulmonary (including but not limited to pulmonary administration by oral inhalation), and intranasal administration. A particularly preferred route of administration for use in the methods described herein is oral administration. In certain embodiments, ebselen is administered by intravenous bolus infusion followed by continuous intravenous infusion.

The daily dose of a pharmaceutical composition including ebselen may be varied over a wide range from about 1 mg to about 3000 mg; preferably, the dose will be in the range of from about 200 mg to about 2000 mg per day, or from about 400 mg to about 2000 mg per day for an average human. For oral administration, the compositions are preferably provided in the form of tablets or capsules containing, 25, 50, 100, 150, 200, 300, 400, 500, 600, 700, 800, 900, 1000, 1250, 1500, 1750, or 2000 milligrams of the ebselen active ingredient for the symptomatic adjustment of the dosage to the subject to be treated.

Advantageously, the ebselen compound may be administered in a single daily dose or the total daily dosage may be administered in divided doses of two, three or four times daily. In some embodiments, the total daily dosage is administered in divided doses of one to three daily. In some embodiments, the total daily dose of ebselen is 400 mg. In some embodiments, the total daily dose of ebselen is 600 mg. In some embodiments, the total daily dose of ebselen is 800 mg. In some embodiments, the ebselen is administered twice per day (BID). In some embodiments, the twice per day (BID) dose of ebselen is 400 mg. In some embodiments, the twice per day (BID) dose of ebselen is 600 mg. In some embodiments, the twice per day (BID) dose of ebselen is 800 mg.

The therapeutically effective dose for ebselen or a pharmaceutical composition thereof may vary according to the desired effect. Therefore, optimal dosages to be administered may be readily determined by those skilled in the art, and may vary with the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this disclosure.

In certain embodiments, the ebselen is administered for 2-14 days. In various embodiments, the ebselen is administered for 2 days, 3 days, 4 days, 5 days, 6 days, 7 days, 8 days, 9 days, 10 days, 11 days, 12 days, 13 days, 14 days, 21 days, 29 days, or longer. In some embodiments, the ebselen is administered for seven days or more. In some embodiments, the ebselen is administered for 7 to 14 days. In some embodiments, the ebselen is administered for 14 to 21 days. In some embodiments, the ebselen is administered for 21 days or more. ebselen is administered for 21 to 28 days. In some embodiments, the ebselen is administered for 28 days or more, such as 6 weeks or more, 2 months or more, or 3 months or more.

5.2.4. Additional Agents

In some embodiments, the methods of the present disclosure further comprise administering an effective amount of at least one second therapeutic agent.

In certain embodiments, one or more corticosteroids may be administered to the patient, either prior to, concurrently, or post-administration of ebselen.

In certain embodiments, the second therapeutic agent is selected from the group consisting of an antiviral agent, an antibacterial agent, an angiotensin receptor blocker (ARB), an IL-6 inhibitor, hydroxychloroquine, chloroquine, and COVID-19 immune serum or plasma.

In certain embodiments, anticoagulants are not administered to the patient in addition to ebselen. In particular embodiments, low molecular weight heparin (LMWH) is not administered to the patient undergoing ebselen treatment. In certain embodiments, unfractionated heparin is administered to the patient undergoing ebselen treatment.

5.2.4.1 Anti-Viral Agents

In some embodiments, the method of the present disclosure further comprises administering an effective amount of an anti-viral agent.

In some embodiments, the anti-viral agent is selected from the group consisting of: favipiravir, favilavir, remdesivir, galidesivir and a combination of lopinavir and ritonavir.

In particular embodiments, the anti-viral agent is favipiravir.

In particular embodiments, the anti-viral agent is remdesivir.

In particular embodiments, the anti-viral agent is galidesivir.

In particular embodiments, the anti-viral agent is a combination of lopinavir and ritonavir.

5.2.4.2 Antibacterial Agents

In some embodiments, the method of the present disclosure further comprises administering an antibacterial agent. In some embodiments, the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin. In certain embodiments, the antibacterial agent is azithromycin.

5.2.4.3 Angiotensin Receptor Blocker (ARB)

In some embodiments, the methods herein further comprise administering an ARB.

In particular embodiments, the ARB is selected from losartan, valsartan, azilsartan, candesartan, eprosartan, irgesartan, olmesartan, and telmisartan.

5.2.4.4 IL-6 Antagonists

In certain embodiments, the patient is further administered an IL-6 antagonist. In some embodiments, the IL-6 inhibitor or antagonist is selected from the group consisting of: an anti-IL-6 receptor antibody or an antigen binding fragment thereof; an anti-IL-6 antibody or an antigen binding fragment thereof; and a JAK/STAT inhibitor.

5.2.4.4.1 Anti-IL-6 Receptor Antibodies

In various embodiments, the IL-6 antagonist is an anti-IL-6 receptor (anti-IL-6R) antibody or antigen-binding fragment or derivative thereof.

In typical embodiments, the anti-IL-6R reduces the biological activity of IL-6 receptor.

In some embodiments, the IL-6 antagonist is an anti-IL-6R monoclonal antibody. In some embodiments, the IL-6 antagonist is a polyclonal composition comprising a plurality of species of anti-IL-6R antibodies, each of the plurality having unique CDRs.

In some embodiments, the anti-IL-6R antibody is a Fab, Fab′, F(ab′)2, Fv, scFv, (scFv)2, single chain antibody molecule, dual variable domain antibody, single variable domain antibody, linear antibody, or V domain antibody.

In some embodiments, the anti-IL-6R antibody comprises a scaffold. In certain embodiments, the scaffold is Fc, optionally human Fc. In some embodiments, the anti-IL-6R antibody comprises a heavy chain constant region of a class selected from IgG, IgA, IgD, IgE, and IgM. In certain embodiments, the anti-IL-6R antibody comprises a heavy chain constant region of the class IgG and a subclass selected from IgG1, IgG2, IgG3, and IgG4.

In some embodiments, the IL-6 antagonist is immunoconjugate or fusion protein comprising an IL-6R antigen-binding fragment.

In some embodiments, the antibody is bispecific or multispecific, with at least one of the antigen-binding portions having specificity for IL-6 receptor.

In some embodiments, the antibody is fully human. In some embodiments, the antibody is humanized. In some embodiments, the antibody is chimeric and has non-human V regions and human C region domains. In some embodiments, the antibody is murine.

In typical embodiments, the anti-IL-6R antibody has a KD for binding human IL-6 receptor of less than 100 nM. In some embodiments, the anti-IL-6R antibody has a KD for binding human IL-6 receptor of less than 75 nM, 50 nM, 25 nM, 20 nM, 15 nM, or 10 nM. In particular embodiments, the anti-IL-6R antibody has a KD for binding human IL-6 receptor of less than 5 nM, 4 nM, 3 nM, or 2 nM. In selected embodiments, the anti-IL-6R antibody has a KD for binding human IL-6 receptor of less than 1 nM, 750 pM, or 500 pM. In specific embodiments, the anti-IL-6R antibody has a KD for binding human IL-6 receptor of no more than 500 pM, 400 pM, 300 pM, 200 pM, or 100 pM.

In typical embodiments, the anti-IL-6R antibody has an elimination half-life following intravenous administration of at least 7 days. In certain embodiments, the anti-IL-6R antibody has an elimination half-life of at least 14 days, at least 21 days, or at least 30 days.

In some embodiments, the anti-IL-6R antibody has a human IgG constant region with at least one amino acid substitution that extends serum half-life as compared to the unsubstituted human IgG constant domain.

Tocilizumab and Derivatives

In certain embodiments, the anti-IL-6R antibody or antigen-binding portion thereof comprises all six CDRs of tocilizumab. In particular embodiments, the antibody or antigen-binding portion thereof comprises the tocilizumab heavy chain V region and light chain V region. In specific embodiments, the antibody is the full-length tocilizumab antibody.

In various embodiments, the anti-IL-6R antibody is a derivative of tocilizumab.

In some embodiments, the tocilizumab derivative includes one or more amino acid substitutions in the tocilizumab heavy and/or light chain V regions.

In certain embodiments, the tocilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the tocilizumab anti-IL-6R antibody, while retaining specificity for human IL-6 receptor.

In certain embodiments, the tocilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of tocilizumab. The percent sequence identity is determined using BLAST algorithms using default parameters.

In certain embodiments, the tocilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of tocilizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

Sarilumab and Derivatives

In certain embodiments, the anti-IL-6R antibody or antigen-binding portion thereof comprises all six CDRs of sarilumab. In particular embodiments, the antibody or antigen-binding portion thereof comprises the sarilumab heavy chain V region and light chain V region. In specific embodiments, the antibody is the full-length sarilumab antibody.

In various embodiments, the anti-IL-6R antibody is a derivative of sarilumab.

In some embodiments, the sarilumab derivative includes one or more amino acid substitutions in the sarilumab heavy and/or light chain V regions.

In certain embodiments, the sarilumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the sarilumab anti-IL-6R antibody, while retaining specificity for human IL-6 receptor.

In certain embodiments, the sarilumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of sarilumab. The percent sequence identity is determined using BLAST algorithms using default parameters.

In certain embodiments, the sarilumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of sarilumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

Vobarilizumab and Derivatives

In certain embodiments, the anti-IL-6R antibody or antigen-binding portion thereof comprises all six CDRs of vobarilizumab. In particular embodiments, the antibody or antigen-binding portion thereof comprises the vobarilizumab heavy chain V region and light chain V region. In specific embodiments, the antibody is the full-length vobarilizumab antibody.

In various embodiments, the anti-IL-6R antibody is a derivative of vobarilizumab.

In some embodiments, the vobarilizumab derivative includes one or more amino acid substitutions in the vobarilizumab heavy and/or light chain V regions.

In certain embodiments, the vobarilizumab derivative comprises fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, fewer than 2 amino acid substitutions, or 1 amino acid substitution relative to the original VH and/or VL of the vobarilizumab anti-IL-6R antibody, while retaining specificity for human IL-6 receptor.

In certain embodiments, the vobarilizumab derivative comprises an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the VH and VL domain of vobarilizumab. The percent sequence identity is determined using BLAST algorithms using default parameters.

In certain embodiments, the vobarilizumab derivative comprises an amino acid sequence in which the CDRs comprise an amino acid sequence that is at least 45%, at least 50%, at least 55%, at least 60%, at least 65%, at least 70%, at least 75%, at least 80%, at least 85%, at least 90%, at least 95%, or at least 99% identical to the amino acid sequence of the respective CDRs of vobarilizumab. The percent sequence identity is determined using BLAST algorithms using default parameters.

In certain embodiments, the VH and/or VL CDR derivatives comprise conservative amino acid substitutions at one or more predicted nonessential amino acid residues (i.e., amino acid residues which are not critical for the antibody to specifically bind to human IL 6 receptor).

Other Anti-IL-6R Antibodies and Derivatives

In certain embodiments, the anti-IL-6R antibody or antigen-binding portion thereof comprises all six CDRs of an antibody selected from the group consisting of: SA237 (Roche), NI-1201 (NovImmune), and an antibody described in US 2012/0225060. In particular embodiments, the antibody or antigen-binding portion thereof comprises the heavy chain V region and light chain V region of an antibody selected from the group consisting of: SA237 (Roche), NI-1201 (NovImmune), and an antibody described in US 2012/0225060. In specific embodiments, the antibody is a full-length selected from the group consisting of: SA237 (Roche), NI-1201 (NovImmune), and an antibody described in US 2012/0225060.

In various embodiments, the anti-IL-6R antibody is a derivative of an antibody selected from the group consisting of: SA237 (Roche), NI-1201 (NovImmune), or an antibody described in US 2012/0225060.

Anti-IL-6:IL-6R Complex Antibodies

In various embodiments, the IL-6 antagonist is an antibody specific for the complex of IL-6 and IL-6R. In certain embodiments, the antibody has the six CDRs of an antibody selected from those described in US 2011/0002936, which is incorporated herein by reference in its entirety.

5.2.4.4.2 Anti-IL-6 Antibodies

In various embodiments, the IL-6 antagonist is an anti-IL-6 antibody or antigen-binding fragment thereof.

In typical embodiments, the anti-IL-6 antibody or antigen-binding fragment thereof neutralizes the biological activity of human IL-6. In some embodiments, the neutralizing antibody prevents binding of IL-6 to the IL-6 receptor. In certain embodiments, the neutralizing antibody prevents binding of IL-6 to the soluble IL-6 receptor. In certain embodiments, the neutralizing antibody prevents binding of IL-6 to the membrane-bound IL-6 receptor. In certain embodiments, the neutralizing antibody prevents binding of IL-6 to both the soluble IL-6 receptor and the membrane-bound IL-6 receptor.

In some embodiments, the IL-6 antagonist is an anti-IL-6 monoclonal antibody. In some embodiments, the IL-6 antagonist is a polyclonal composition comprising a plurality of species of anti-IL-6 antibodies, each of the plurality having unique CDRs.

In some embodiments, the anti-IL-6 antibody is selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza). In some embodiments, the antigen-binding fragment is a fragment of an antibody selected from the group consisting of: ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

5.2.4.4.3 IL-6 Antagonist Peptides

In various embodiments, the IL-6 antagonist is an antagonist peptide.

In certain embodiments, the IL-6 antagonist is C326 (an IL-6 inhibitor by Avidia, also known as AMG220), or FE301, a recombinant protein inhibitor of IL-6 (Ferring International Center S.A., Conaris Research Institute AG). In some embodiments, the anti-IL-6 antagonist comprises soluble gp130, FE301 (Conaris/Ferring).

5.2.4.4.4 JAK and STAT Inhibitors

In various embodiments, the IL-6 antagonist is an inhibitor of the JAK signaling pathway. In some embodiments, the JAK inhibitor is a JAK1-specific inhibitor. In some embodiments, the JAK inhibitor is a JAK3-specific inhibitor. In some embodiments, the JAK inhibitor is a pan-JAK inhibitor. In certain embodiments, the JAK inhibitor is selected from the group consisting of tofacitinib (Xeljanz), decernotinib, ruxolitinib, upadacitinib, baricitinib, filgotinib, lestaurtinib, pacritinib, peficitinib, momelotinib, INCB-039110, ABT-494, INCB-047986 and AC-410.

In various embodiments, the IL-6 antagonist is a STAT3 inhibitor. In a specific embodiment, the inhibitor is AZD9150 (AstraZeneca, Isis Pharmaceuticals), a STAT3 antisense molecule.

In typical embodiments, small molecule JAK inhibitors and STAT inhibitors are administered orally.

In various embodiments, the inhibitor is administered once or twice a day at an oral dose of 0.1-1 mg, 1-10 mg, 10-20 mg, 20-30 mg, 30-40 mg, or 40-50 mg. In some embodiments, the inhibitor is administered once or twice a day at a dose of 50-60 mg, 60-70 mg, 70-80 mg, 80-90 mg, or 90-100 mg. In some embodiments, the inhibitor is administered at a dose of 0.1, 0.5, 1, 2, 5, 10, 15, 20, 25, 30, 35, 40, 45, or 50 mg PO once or twice a day. In some embodiments, the inhibitor is administered at a dose of 75 mg or 100 mg PO once or twice a day.

5.2.4.5 Hydroxychloroquine and Chloroquine

In some embodiments, the method further comprises administering an anti-malarial agent. In certain embodiments, the anti-malarial agent is hydroxychloroquine. In certain embodiments, the anti-malarial agent is chloroquine.

5.2.4.6 COVID-19 Immune Serum or Plasma

In some embodiments, the method further comprises administering a COVID-19 immune serum or plasma, or a composition comprising isolated or recombinantly expressed anti-SARS-CoV-2 antibodies having sequences derived from COVID-19 immune serum or plasma.

5.2.5. Post-Treatment Reduction of IL-6 and C-Reactive Protein (CRP)

In some embodiments, the administration of an effective amount of ebselen reduces the patient's free serum IL-6 levels below pre-treatment levels. In various embodiments, the dosage regimen is adjusted to achieve a reduction in the patient's free serum IL-6 levels below pre-treatment levels.

In some embodiments, the free serum IL-6 level is decreased by at least 10% as compared to pre-treatment levels. In various embodiments, the free serum IL-6 level is decreased by at least 20%, 30%, 40%, 50%, 60%, 70%, 80%, or 90% as compared to pre-treatment levels. In certain embodiments, the free serum IL-6 level is decreased by at least 20% as compared to pre-treatment levels. In certain embodiments, the free serum IL-6 level is decreased by at least 30% as compared to pre-treatment levels. In certain embodiments, the free serum IL-6 level is decreased by at least 40% as compared to pre-treatment levels. In certain embodiments, the free serum IL-6 level is decreased by at least 50% as compared to pre-treatment levels. In certain embodiments, the free serum IL-6 level is decreased by at least 60% as compared to pre-treatment levels. In certain embodiments, the free serum IL-6 level is decreased by at least 70% as compared to pre-treatment levels. In certain embodiments, the free serum IL-6 level is decreased by at least 80% as compared to pre-treatment levels. In certain embodiments, the free serum IL-6 level is decreased by at least 90% as compared to pre-treatment levels.

In some embodiments, the administration of an effective amount of ebselen, reduces the patient's serum CRP levels below pre-treatment levels. In various embodiments, the dosage regimen is adjusted to achieve a reduction in the patient's serum CRP levels below pre-treatment levels.

In some embodiments, the post-treatment CRP level is no more than 45 mg/L. In certain embodiments, the post-treatment CRP level is no more than 40 mg/L. In certain embodiments, the post-treatment CRP level is no more than 30 mg/L. In certain embodiments, the post-treatment CRP level is no more than 20 mg/L. In certain embodiments, the post-treatment CRP level is no more than 10 mg/L. In certain embodiments, the post-treatment CRP level is no more than 5 mg/L. In certain embodiments, the post-treatment CRP level is no more than 2.5 mg/L. In certain embodiments, the post-treatment CRP level is no more than 2 mg/L. In certain embodiments, the post-treatment CRP level is no more than 1 mg/L.

In some embodiments, the CRP level is decreased by at least 10% as compared to pre-treatment levels. In various embodiments, the CRP level is decreased by at least 20%, 30%, 40%, 50%, 60%, 70%, 80%, or 90% as compared to pre-treatment levels. In certain embodiments, the CRP level is decreased by at least 20% as compared to pre-treatment levels. In certain embodiments, the CRP level is decreased by at least 30% as compared to pre-treatment levels. In certain embodiments, the CRP level is decreased by at least 40% as compared to pre-treatment levels. In certain embodiments, the CRP level is decreased by at least 50% as compared to pre-treatment levels. In certain embodiments, the CRP level is decreased by at least 60% as compared to pre-treatment levels. In certain embodiments, the CRP level is decreased by at least 70% as compared to pre-treatment levels. In certain embodiments, the CRP level is decreased by at least 80% as compared to pre-treatment levels. In certain embodiments, the CRP level is decreased by at least 90% as compared to pre-treatment levels.

5.2.6. Other Post-Treatment Endpoints

In some embodiments, administering an effective amount of ebselen to the patient prevents a hyperinflammatory response in the patient. In some embodiments, the dosage regimen is adjusted to prevent a hyperinflammatory response in the patient.

In some embodiments, administering an effective amount of ebselen to the patient results in a reduction in body temperature. In some embodiments, the patient, post-treatment with an effective amount of ebselen, has a body temperature of 37.5° C. or below. In some embodiments, the patient, post-treatment with an effective amount of ebselen, has a body temperature ranging from of 36 to 37.5° C.

In some embodiments, administering an effective amount of ebselen to the patient results in a reduction in the risk of respiratory morbidity and mortality. In some embodiments, the dose is adjusted to reduce the risk of respiratory morbidity and mortality.

In some embodiments, administering an effective amount of ebselen to the patient results in a reduction in the patient's need for supplemental oxygen. In some embodiments, the dose is adjusted to reduce the patient's need for supplemental oxygen.

In some embodiments, administering an effective amount of ebselen to the patient results in eliminating the patient's need for assisted ventilation. In some embodiments, the dose is adjusted to eliminate the patient's need for assisted ventilation.

Additionally, any of the primary and/or secondary endpoints described herein can be met by administering an effective amount of ebselen as described herein.

### 5.3. Kits and Compositions

Additionally, certain components or embodiments of the compositions can be provided in a kit. For example, the ebselen composition, as well as the related buffers or other components related to administration can be provided in separate containers and packaged as a kit, alone or along with separate containers of any of the other agents from any pre-conditioning or post-conditioning steps, and optional instructions for use. In some embodiments, the kit may comprise ampoules, disposable syringes, capsules, vials, tubes, or the like. In some embodiments, the kit may comprise a single dose container or multiple dose containers comprising the embodiments herein. In some embodiments, each dose container may contain one or more unit doses. In some embodiments, the kit may include an applicator for localized or parenteral routes of administration. In some embodiments, the kits include all components needed for the various stages of treatment. In some embodiments, the compositions may have preservatives or be preservative-free (for example, in a single-use container). In some embodiments, the kit may comprise materials for intravenous administration. In some embodiments, the kit may comprise protamine in a separate container, which can be administered to rapidly neutralize anticoagulation due to unfractionated heparin (UFH). In some embodiments, the kit may comprise a nebulizer in a separate container, which can aerosolize the ebselen composition for rapid and direct delivery to the lung.

Also provided are pharmaceutical compositions including ebselen, at least one second therapeutic agent (e.g., as described herein), and a pharmaceutically acceptable diluent or excipient. The composition can include ebselen and an antiviral agent. In some embodiments, the composition includes remdesivir. In some embodiments, the composition includes favilavir. In some embodiments, the composition includes galidesivir. In some embodiments, the composition includes favipiravir.

### 5.4. Additional Embodiments

Additional embodiments of this disclosure are set forth in the following clauses.

Clause 1. A method of treating a patient who has or is at risk for acute lung injury (ALI), acute respiratory distress syndrome (ARDS), ALI with concomitant pneumonia, or ARDS with concomitant pneumonia, comprising: administering an effective amount of ebselen to a patient who has or is at risk for acute lung injury (ALI), acute respiratory distress syndrome (ARDS), ALI with concomitant pneumonia, or ARDS with concomitant pneumonia.

Clause 2. A method of treating a patient who has a confirmed or suspected viral lung infection, comprising: administering a therapeutically effective amount of ebselen to a patient who is suffering from, or suspected of having, a viral lung infection.

Clause 3. A method of treating a patient who has or is at risk for cytokine release syndrome (CRS), comprising: administering an effective amount of ebselen to a patient who has, or is at risk for, CRS.

Clause 4. The method of any one of clauses 1-3, wherein the patient has a viral infection.

Clause 5. The method of clause 4, wherein the infection is by a virus selected from coronavirus, influenza virus, rhinovirus, respiratory syncytial virus, metapneumovirus, adenovirus, and boca virus.

Clause 6. The method of clause 5, wherein the virus is a coronavirus selected from coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19).

Clause 7. The method of clause 6, wherein the coronavirus is SARS-CoV-2 (COVID-19).

Clause 8. The method of any one of clauses 1 to 8, wherein the patient has or is at risk for ALI.

Clause 9. The method of any one of clauses 1 to 8, wherein the patient has or is at risk of ARDS.

Clause 10. The method of any one of clauses 1 to 8, wherein the patient has or is at risk for ALI with concomitant pneumonia or ARDS with concomitant pneumonia.

Clause 11. The method of any one of clauses 1 to 10, wherein the patient is asymptomatic or has mild symptoms of viral infection within 1 to 5 days prior to treatment.

Clause 12. The method of clause 11, wherein prior to treatment the patient is diagnosed as nCoV2 positive.

Clause 13. The method of clause 11 or 12, wherein the patient is naïve to treatment with an anti-viral agent (e.g., remdesivir, favilavir or galidesivir), hydroxychloroquine or azithromycin.

Clause 14. The method of any one of clauses 11 to 13, wherein the ebselen is administered at 1 to 3 doses per day.

Clause 15. The method of clause 14, wherein the daily dose is 400 mg to 2000 mg.

Clause 16. The method of clause 15, wherein the ebselen is administered twice per day (BID).

Clause 17. The method of clause 16, wherein the twice per day (BID) dose is 400 mg.

Clause 18. The method of clause 16, wherein the twice per day (BID) dose is 600 mg.

Clause 19. The method of clause 16, wherein the twice per day (BID) dose is 800 mg.

Clause 20. The method of any one of clauses 11-19, wherein ebselen is administered for seven days or more (e.g., 7 days or 14 days or 21 days).

Clause 21. The method of any one of clauses 11-20, further comprising assessing viral load in a sample of the patient.

Clause 22. The method of clause 21, wherein the viral load of the patient is assessed daily.

Clause 23. The method of clause 21 or 22, further comprising adjusting the daily dose of ebselen administered to the patient when the viral load is assessed as reduced by less than 1-log after 7 days of treatment.

Clause 24. The method of any one of clauses 21-23, wherein the viral load of the patient is assessed as reduced by 1-log or more, 2-log or more, or 3-log or more within or after 7 days of treatment.

Clause 25. The method of any one of clauses 1 to 10, wherein prior to treatment the patient has mild or moderate symptoms of viral infection.

Clause 26. The method of clause 25, wherein prior to treatment the patient is diagnosed as having SARS-CoV-2 (COVID-19).

Clause 27. The method of clause 25 or 26, wherein the patient is naïve to treatment with an anti-viral agent (e.g., remdesivir, favilavir or galidesivir).

Clause 28. The method of clause 25 or 26, wherein the patient is naïve to treatment with hydroxychloroquine or azithromycin.

Clause 29. The method of any one of clauses 25 to 28, wherein the ebselen is administered at 1 to 3 doses per day.

Clause 30. The method of clause 29, wherein the daily dose is 400 mg to 2000 mg.

Clause 31. The method of clause 30, wherein the ebselen is administered twice per day (BID).

Clause 32. The method of clause 31, wherein the twice per day (BID) dose is 400 mg.

Clause 33. The method of clause 31, wherein the twice per day (BID) dose is 600 mg.

Clause 34. The method of clause 31, wherein the twice per day (BID) dose is 800 mg.

Clause 35. The method of any one of clauses 25 to 34, wherein ebselen is administered for 14 to 21 days.

Clause 36. The method of any one of clauses 25 to 34, wherein ebselen is administered for 21 days or more.

Clause 37. The method of any one of clauses 25 to 36, wherein administering an effective amount of ebselen to the patient results in a reduction in the risk of respiratory morbidity and mortality.

Clause 38. The method of any one of clauses 25 to 36, wherein administering an effective amount of ebselen to the patient results in a reduction in the patient's need for supplemental oxygen.

Clause 39. The method of any one of clauses 25 to 36, wherein administering an effective amount of ebselen to the patient results in a reduction in the risk of assisted ventilation.

Clause 40. The method of any one of clauses 25 to 36, wherein administering an effective amount of ebselen to the patient results in a reduction in the risk of ARDS.

Clause 41. The method of any one of clauses 1 to 10, wherein the patient has moderate or severe symptoms of viral infection.

Clause 42. The method of clause 41, wherein prior to treatment the patient is diagnosed as having moderate or severe SARS-CoV-2 (COVID-19).

Clause 43. The method of clause 41 or 42, wherein the patient is naïve to treatment with an anti-viral agent (e.g., remdesivir, favilavir or galidesivir).

Clause 44. The method of clause 41 or 42, wherein the patient is naive to treatment with hydroxychloroquine or azithromycin.

Clause 45. The method of clause 41 or 42, wherein the patient is refractory to treatment with an anti-viral agent.

Clause 46. The method of clause 45, wherein the anti-viral agent is remdesivir, favilavir or galidesivir.

Clause 47. The method of clause 41 or 42, wherein the patient is refractory to treatment with hydroxychloroquine or azithromycin.

Clause 48. The method of any one of clauses 41 to 47, wherein the ebselen is administered at 1 to 3 doses per day.

Clause 49. The method of clause 48, wherein the daily dose is 400 mg to 2000 mg.

Clause 50. The method of clause 49, wherein the ebselen is administered twice per day (BID).

Clause 51. The method of clause 50, wherein the twice per day (BID) dose is 400 mg.

Clause 52. The method of clause 50, wherein the twice per day (BID) dose is 600 mg.

Clause 53. The method of clause 50, wherein the twice per day (BID) dose is 800 mg.

Clause 54. The method of any one of clauses 41-53, wherein ebselen is administered for 14 to 28 days, such as 14 days or 21 to 28 days.

Clause 55. The method of any one of clauses 41-53, wherein ebselen is administered for 28 days or more.

Clause 56. The method of any one of clauses 41-53, wherein prior to treatment the patient needs assisted ventilation, or is on a ventilator.

Clause 57. The method of any one of clauses 41-53, wherein the ebselen is administered via nasogastric (ng) tube.

Clause 58. The method of any one of clauses 41-57, wherein administering an effective amount of ebselen to the patient results in a reduction in the risk of respiratory failure.

Clause 59. The method of any one of clauses 41-57, wherein administering an effective amount of ebselen to the patient results in eliminating the patient's need for assisted ventilation.

Clause 60. The method of any one of clauses 41-57, further comprising adjusting the administered dose of ebselen to eliminate the patient's need for assisted ventilation.

Clause 61. The method of any one of clauses 1 to 56, wherein the ebselen is administered orally.

Clause 62. The method of clause 61, wherein ebselen is formulated for oral administration in a solid dosage form.

Clause 63. The method of clause 57, wherein the solid dosage form is a capsule.

Clause 64. The method of any one of clauses 1 to 51, wherein the ebselen is administered intravenously or by inhalation.

Clause 65. The method of any one of clauses 1 to 64, wherein the patient is not hospitalized.

Clause 66. The method of any one of clauses 1 to 64, wherein the patient is hospitalized.

Clause 67. The method of clause 66, wherein the patient is not on a ventilator.

Clause 68. The method of clause 67, wherein the administration of ebselen reduces or eliminates the patient's need for assisted ventilation.

Clause 69. The method of any one of clauses 1 to 68, wherein the patient has a body temperature of greater than 37.5° C. prior to first administration of ebselen.

Clause 70. The method of clause 69, wherein the body temperature of the patient is measured at one or more sites selected from the group consisting of an oral cavity, a rectal cavity, axilla area, and tympanic membrane.

Clause 71. The method of clause 69 or 70 wherein the method reduces the body temperature of the patient below pre-treatment levels.

Clause 72. The method of any one of clauses 1 to 71, wherein the patient has a pre-treatment C-creative protein (CRP) level greater than 2 mg/L.

Clause 73. The method of clause 72, wherein the patient has a pre-treatment CRP level greater than 5 mg/L, greater than 10 mg/L, greater than 20 mg/L, greater than 30 mg/L, or greater than 40 mg/L.

Clause 74. The method of any one of clauses 1 to 73, wherein the method reduces the patient's serum CRP levels below pre-treatment levels.

Clause 75. The method of clause 74, wherein the post-treatment CRP level is no more than 45 mg/L, no more than 40 mg/L, no more than 35 mg/L, no more than 30 mg/L, no more than 20 mg/L, no more than 10 mg/L, no more than 5 mg/L, or no more than 1 mg/L.

Clause 76. The method of any one of clauses 1-75, wherein the method reduces the CRP level by at least 10% (e.g., at least 20%, 30%, 40%, 50%, 60%, 70%, 80%, or 90%) as compared to pre-treatment levels.

Clause 77. The method of any one of clauses 1 to 76, wherein the patient has a pre-treatment free IL-6 level in serum of at least 2 pg/ml.

Clause 78. The method of clause 77, wherein the patient has a pre-treatment free IL-6 level in serum of at least 2.5 pg/ml, 3 pg/ml, 4 pg/ml, 5 pg/ml, 10 pg/ml, 20 pg/ml, 30 pg/ml, 40 pg/ml, 50 pg/ml, 60 pg/ml, 70 pg/ml, 80 pg/ml, 90 pg/ml, 100 pg/ml, 150 pg/ml or 200 pg/ml.

Clause 79. The method of any one of clauses 1 to 78, wherein the method reduces the patient's free IL-6 levels in serum below pre-treatment levels.

Clause 80. The method of clause 79, wherein the free IL-6 level in serum is decreased by at least 10% as compared to pre-treatment levels.

Clause 81. The method of clause 80, wherein the free IL-6 level in serum is decreased by at least 20%, 30%, 40%, 50%, 60%, 70%, 80%, or 90% as compared to pre-treatment levels.

Clause 82. The method of any one of clauses 1 to 81, wherein the patient has a pre-treatment neutrophil-to-lymphocyte ratio (NLR) greater than 2.0.

Clause 83. The method of clause 82, wherein the patient has a pre-treatment NLR greater than 2.8, 2.9, 3.0, 3.1, 3.2, 3.3, 3.4, 3.5, 3.6, 3.7, 3.8, 3.9, or 4.0.

Clause 84. The method of clause 82, wherein the patient has a pre-treatment D-Dimer level that is elevated above baseline.

Clause 85. The method of clause 82, wherein the patient has a pre-treatment sepsis-induced coagulopathy (SIC) total score of 4 or more with total score of prothrombin time and coagulation exceeding 2.

Clause 86. The method of clause 83, wherein the patient has a post-treatment NLR less than 3.18.

Clause 87. The method of clause 86, wherein the administration of ebselen decreases the NLR by at least 10%, 20%, 30%, 40%, 50%, 60%, 70%, 80%, or 90% as compared to pre-treatment levels.

Clause 88. The method of any one of clauses 1 to 87, wherein the patient has a pre-treatment respiration rate on ambient air of fewer than 12 breaths or more than 20 breaths per minute.

Clause 89. The method of clause 88, wherein the method improves the respiration rate of the patient.

Clause 90. The method of clause 89, wherein the patient has a post-treatment respiration rate between 12 to 20 breaths per minute.

Clause 91. The method of any one of clauses 1 to 90, wherein the patient has a pre-treatment oxygen saturation level on ambient air of no more than 93%.

Clause 92. The method of any one of clause 1 to 90, wherein the patient has a pre-treatment oxygen saturation level on ambient air of no more than 85%, 80%, 75%, 70%, 65% or 60%.

Clause 93. The method of any one of clauses 1 to 92, wherein the method improves the oxygen saturation level of the patient on ambient air by 10%, 20%, 30%, 40%, 50%, 60%, 70%, 80%, or 90% as compared to pre-treatment levels.

Clause 94. The method of any one of clauses 1 to 93, wherein the method reduces the patient's need for supplemental oxygen.

Clause 95. The method of any one of clauses 1 to 94, wherein the patient is older than 50, 51, 52, 53, 54, 55, 56, 57, 58, or 59 years of age.

Clause 96. The method of any one of clauses 1 to 95, wherein the patient is older than 60 years of age.

Clause 97. The method of any one of clauses 1 to 94, wherein the patient is younger than 60, 59, 58, 57, 56, 55, 54, 53, 52, 51, or 50 years of age.

Clause 98. The method of any one of clauses 1 to 97, wherein the method further comprises administering an effective amount of at least one second therapeutic agent selected: an antiviral agent, antibacterial agent, an angiotensin receptor blocker (ARB), an IL-6 inhibitor, hydroxychloroquine, chloroquine, and COVID-19 immune serum or plasma.

Clause 99. The method of clause 98, wherein the at least one second therapeutic agent is an antiviral agent.

Clause 100. The method of clause 99, wherein the antiviral agent is favipiravir or galidesivir.

Clause 101. The method of clause 99, wherein the second therapeutic agent is remdesivir.

Clause 102. The method of clause 98, wherein the at least one second therapeutic agent is an antibacterial agent.

Clause 103. The method of clause 102, wherein the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin.

Clause 104. The method of clause 103, wherein the antibacterial agent is azithromycin.

Clause 105. The method of clause 98, wherein the at least one second therapeutic agent is an ARB.

Clause 106. The method of clause 105, wherein the ARB is losartan.

Clause 107. The method of clause 105, wherein the ARB is valsartan.

Clause 108. The method of clause 98, wherein the at least one second therapeutic agent is an IL-6 inhibitor.

Clause 109. The method of clause 108, wherein the IL-6 inhibitor is selected from the group consisting of: an anti-IL-6 receptor antibody or an antigen binding fragment thereof, an anti-IL-6 antibody or an antigen binding fragment thereof, and a JAK/STAT inhibitor.

Clause 110. The method of clause 109, wherein the IL-6 inhibitor is an anti-IL-6 receptor antibody, or antigen binding fragment thereof.

Clause 111. The method of clause 109, wherein the anti-IL-6 receptor antibody is tocilizumab or sarilumab.

Clause 112. The method of clause 109, wherein the IL-6 inhibitor is an anti-IL-6 antibody, or antigen binding fragment thereof.

Clause 113. The method of clause 112, wherein the anti-IL-6 antibody is selected from the group consisting of ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

Clause 114. The method of clause 109, wherein the IL-6 inhibitor is a JAK/STAT inhibitor.

Clause 115. The method of clause 114, wherein the JAK/STAT inhibitor is selected from the group consisting of ruxolotinib, tofacitinib, and baricitinib.

Clause 116. A pharmaceutical composition for treating acute lung injury (ALI), acute respiratory distress syndrome (ARDS), ALI with concomitant pneumonia, or ARDS with concomitant pneumonia, comprising: ebselen; an antiviral agent; and a pharmaceutically acceptable excipient.

Clause 117. The composition of clause 116, wherein the anti-viral agent is selected from remdesivir, favilavir and galidesivir.

Clause 118. The composition of clause 117, wherein the anti-viral agent is remdesivir.

### 6. Examples

6.1. SPI-1005-101, a Phase 1, Randomized Clinical Trial (RCT) in Healthy Subjects

In a Phase 1 study, 32 subjects were randomized to receive either a single oral dose of placebo, or one of four different doses of SPI-1005. There were no deaths or Serious Adverse Events (SAEs) reported. There were no treatment- or dose-related trends in overall adverse effects (AE) incidence when compared to placebo. Pharmacokinetic analysis of ebselen and its metabolites supported twice daily dosing by the oral route of administration.

6.2. Study to Confirm the Safety and Therapeutic Effect of Ebselen for the Early Intervention Treatment or Prevention of COVID-19 in nCoV2 Positive Subjects Having No to Mild COVID-19

6.2.1. Overall Design

A randomized, placebo-controlled, study to confirm safety and therapeutic effect of ebselen in nCoV2 positive patients with no to mild disease.

Eligible participants are diagnosed as nCoV2 positive and enrolled for treatment within 1-5 days of appearance of symptoms. The patient is not treated with another anti-viral therapeutic agent prior to beginning of treatment. The eligible patients have high viral loads (based on respiratory samples) (see viral loads according to Zou et al., “SARS-CoV-2 Viral Load in Upper Respiratory Specimens of Infected Patients”, N Engl J Med 2020; 382:1177-1179; or Pan et al. “Viral load of SARS-CoV-2 in clinical samples”, The Lancet, 20 (4), P411-412, Apr. 1, 2020). Randomization is stratified by age (<60 years or ≥60 years).

Ebselen is dosed as 400 to 600 mg BID for a period of 7-10 days. A Phase 1b study related to Meniere's Disease indicated that the plasma trough concentration of ebselen is approximately 4.1-microM for a 400 mg BID dose after 21 days. Participants receive study intervention for up to 10 days and are followed for 28 days after first dose.

Respiratory samples are taken before treatment begins and daily during treatment to assess nCoV2 viral load daily.

The primary efficacy endpoint is the time to significant reduction in pre-treatment viral load (see Zou et al.; Pan et al.). A significant reduction can refer to a 2-log or greater reduction in a patient's pre-treatment viral load. The therapeutic effect can be observed within 7 to 10 days after beginning of treatment. Higher doses (e.g., 800 mg) or more frequent doses can be administered to achieve a >2 log or 3 log or greater reduction in viral load within 7 days. Other efficacy assessments are also performed and compared to baseline levels.

Follow-up assessments can be performed at 2, 4, and 8 weeks after the start of treatment. When a positive therapeutic effect is observed, dosing can be extended until cure. Extended dosing involves sub-chronic dosing of 4-12 weeks (e.g., 4, 8 or 12 weeks) with assessment of viral load until virus eliminated.

6.2.2. Results

Administration of ebselen is effective to reduce viral load.

6.3. Study to Confirm the Safety and Therapeutic Effect of Ebselen for the Treatment of Mild to Moderate COVID-19 in Subjects at Risk of ALI, or ARDS.

The anti-inflammatory mechanism of action of ebselen is unique and distinct over that of a classic corticosteroid immunosuppressive agent. We did several MIC assays against 18 common microbes to show a lack of interference, to support testing in CF patients. In addition, our Phase 2b study in CF patients with Pseudomonas is continuing without any safety issues. Furthermore, the demonstrated efficacy of ebselen in a Meniere's Disease study to improve auditory function (hearing loss, word recognition and tinnitus) exceed effects reported with corticosteroid treatment in that population.

6.3.1. Overall Design

A randomized, placebo-controlled, study to confirm safety and therapeutic effect of ebselen in COVID-19 patients with mild to moderate disease.

Eligible participants are COVID-19 patients having mild to moderate symptoms of disease.

Ebselen is dosed as 400 to 600 mg BID for a period of 14 to 21 days.

Efficacy is evaluated through clinical examinations, vital signs, oxygen requirements, and laboratory tests. Safety is monitored through collection of AE data and laboratory tests.

Therapeutic benefits include prevention of progression to severe disease (e.g., as defined by ARDS clinical status or need for ventilatory support).

The primary efficacy endpoint is the time to clinical improvement in respiratory status. The endpoint can be defined as defined as maintaining or improving clinical status as measured on the NIAID ordinal scale. The ordinal scale is an assessment of the clinical status of the subject. The scale is as follows: 1) Death; 2) Hospitalized, on invasive mechanical ventilation or extracorporeal membrane oxygenation (ECMO); 3) Hospitalized, on non-invasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

6.3.2. Results

Administration of ebselen is effective to improve in respiratory status and prevent progression to severe disease.

6.4. Study to Confirm the Safety and Therapeutic Effect of Ebselen for the Treatment of Moderate COVID-19 in Subjects at Risk of ALI, or ARDS.

6.4.1. Overall Design

A randomized, placebo-controlled, study to confirm safety and therapeutic effect of ebselen in COVID-19 patients with moderate disease.

Eligible participants are COVID-19 patients having moderate symptoms of disease.

Ebselen is dosed as 400 or 800 mg BID for a period of 7 days with 30-day follow up.

Efficacy is evaluated through clinical examinations, vital signs, oxygen requirements, and laboratory tests. Safety is monitored through collection of AE data and laboratory tests.

Therapeutic benefits include prevention of progression to severe disease (e.g., as defined by ARDS clinical status or need for ventilatory support).

The primary efficacy endpoint is the number of participants with treatment-related adverse events. Another endpoint is the time to clinical improvement in respiratory status. The endpoint can be defined as defined as maintaining or improving clinical status as measured on the WHO ordinal scale for Clinical Improvement (time frame 30 days). Scale is 0-8 where higher score is worse outcome. An additional endpoint is degree of supplemental oxygen (time frame 30 days), where respiratory status is assessed by degree of supplemental oxygen (e.g. mask oxygen, mechanical ventilation). An additional endpoint is peripheral oxygen saturation (SpO₂) (time frame 30 days), where peripheral oxygen saturation is measured by pulse oximetry.

The ordinal scale is an assessment of the clinical status of the subject. The scale is as follows: 1) Death; 2) Hospitalized, on invasive mechanical ventilation or extracorporeal membrane oxygenation (ECMO); 3) Hospitalized, on non-invasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

Inclusion Criteria:
Adults ≥18 years of age;
Positive nCoV2 PCR test by nasopharyngeal, oral, saliva, or respiratory sample;
Clinical signs, symptoms, and respiratory status consistent with moderate COVID-19;
Respiratory status of SpO2>94% and respiratory rate <24 breaths/min without supplemental oxygen;
Score of 3 on the WHO Ordinal Scale (hospitalized, no oxygen treatment);
Symptom onset ≤3 days of study enrollment; and
Subject is in-patient at time of randomization to study treatment Subject or legally authorized representative is willing and able to provide informed consent.

Exclusion Criteria:
Female patients who are pregnant or breastfeeding;
Participation in another interventional investigational drug or device study concurrently or within 30 days prior to study consent;
Patients with impaired hepatic or renal function; and
Subject has any other illness or condition that, in the opinion of the investigator, would prohibit the subject from participating.

6.4.2. Results

Administration of ebselen is effective to improve in respiratory status and prevent progression to severe disease.

6.5. Study to Confirm the Safety and Therapeutic Effect of Ebselen for the Treatment of Moderate to Severe COVID-19 in Subjects at High Risk of Respiratory Failure

6.5.1. Overall Design

Severe COVID-19 patients at risk of or having ARDS will not likely be helped with the addition of other putative anti-viral treatments such as HCQ and AZTH, and are at significant risk for such interventions since they both have QT prolongation issues.

Dosages and dose schedules (i.e. ×21 days) that appear to improve lung functioning test (LFTs) (i.e. FEV1 (forced expiratory volume in one second)) in our ongoing separate study of cystic fibrosis (CF) patients, are applied in this study.

A randomized, placebo-controlled, Phase 1/2 study to confirm safety and therapeutic effect of ebselen in adults with moderate to severe COVID-19 who are at high risk of respiratory failure is conducted.

Eligible participants are hospitalized with laboratory-confirmed SARS-CoV-2, with a resting oxygen saturation (by pulse oximetry) of ≤93% on ambient air are randomized 1:1 to receive ebselen or placebo for up to 21 to 28 days; both groups also receive best supportive care (as determined by the investigator) as background therapy. Randomization is stratified by baseline NIAID score (3 or 4) and by age (<60 years or ≥60 years). Note that the viral load in the more advanced patient is already going down and that CRS can lead to ARDS and death.

Ebselen is dosed as 400 to 600 mg BID for a period of up to 21 to 28 days. For those patients who are intubated or unable to receive ebselen by oral administration (po), the dose of ebselen powder is suspended and delivered by nasogastric (ng) tube.

Efficacy is evaluated through clinical examinations, vital signs, oxygen requirements, and laboratory tests. Safety is monitored through collection of AE data and laboratory tests.

Therapeutic benefits include discontinuation of assisted ventilation. The primary efficacy endpoint is the time to clinical improvement. Time to clinical improvement can be defined as time to at least a 2-grade improvement from baseline on the NIAID ordinal scale. The ordinal scale is an assessment of the clinical status of the subject. The scale is as follows: 1) Death; 2) Hospitalized, on invasive mechanical ventilation or extracorporeal membrane oxygenation (ECMO); 3) Hospitalized, on non-invasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

Secondary endpoints include clinical status assessed by the NIAID ordinal scale at fixed time points, time to hospital discharge or National Early Warning Score (NEWS) of ≤2 maintained for 24 hours, time to resolution of fever for 48 hours without antipyretics, number of ventilator-free days, all-cause mortality, and changes in CRP, d-dimer, serum ferritin, LDH, HMGB1, IL-6, TNFα, PF4, and SAA levels. Safety, including the incidence of AEs, severe AEs, and SAEs, is assessed throughout the study.

6.5.2. Results

Administration of ebselen is effective to reduce time to at least a 2-grade improvement from baseline on the NIAID ordinal scale.

6.6. Study to Confirm the Safety and Therapeutic Effect of Ebselen for the Treatment of Severe COVID-19 in Subjects at Risk of ALI, or ARDS.

6.6.1. Overall Design

A randomized, placebo-controlled, study to confirm safety and therapeutic effect of ebselen in COVID-19 patients with severe disease.

Eligible participants are COVID-19 patients having severe symptoms of disease.

Ebselen is dosed as 400 or 800 mg BID for a period of 14 days with 30-day follow up.

Efficacy is evaluated through clinical examinations, vital signs, oxygen requirements, and laboratory tests. Safety is monitored through collection of AE data and laboratory tests.

The primary efficacy endpoint is the number of participants with treatment-related adverse events. Another endpoint is the time to clinical improvement in respiratory status. The endpoint can be defined as defined as maintaining or improving clinical status as measured on the WHO ordinal scale for Clinical Improvement (time frame 30 days). Scale is 0-8 where higher score is worse outcome. An additional endpoint is degree of supplemental oxygen (time frame 30 days), where respiratory status is assessed by degree of supplemental oxygen (e.g. mask oxygen, mechanical ventilation). An additional endpoint is peripheral oxygen saturation (SpO₂) (time frame 30 days), where peripheral oxygen saturation is measured by pulse oximetry.

The ordinal scale is an assessment of the clinical status of the subject. The scale is as follows: 1) Death; 2) Hospitalized, on invasive mechanical ventilation or extracorporeal membrane oxygenation (ECMO); 3) Hospitalized, on non-invasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

Inclusion Criteria:
Adults ≥18 years of age;
Positive nCoV2 PCR test by nasopharyngeal, oral, saliva, or respiratory sample;
Clinical signs, symptoms, and respiratory status consistent with moderate COVID-19;
Severe disease: requiring mechanical ventilation or oxygen, a SpO₂≤94% on room air, or tachypnoea (respiratory rate ≥24 breaths/min)
Symptom onset ≤7 days of study enrollment; and
Subject is in-patient at time of randomization to study treatment
Subject or legally authorized representative is willing and able to provide informed consent.

Exclusion Criteria:
Female patients who are pregnant or breastfeeding;
Participation in another interventional investigational drug or device study concurrently or within 30 days prior to study consent;
Patients with impaired hepatic or renal function; and
Subject has any other illness or condition that, in the opinion of the investigator, would prohibit the subject from participating.

6.6.2. Results

Administration of ebselen is effective to improve in respiratory status, and/or reduce time to at least a 2-grade improvement from baseline on the WHO ordinal scale.

6.7. Ebselen Shows Reduction of Viral Load In Vitro in Kidney Cells.

Following the protocol for the image based assay of Jin et al. Vero E6 cells (purchased from ATCC) were transfected with SARS-CoV-2 and then incubated with ebselen, added in a solution of either DMSO or EtOH, at concentrations ranging from 100 nM to 1000 μM or remdesivir at concentrations ranging from 1 nM to 10 μM.

6.7.1. Results

TABLE 1
Reduction in viral load in Vero E6 cells

| Compound | EC50 μM | CC50 μM |
|---|---|---|
| ebselen (DMSO) | 21.3 | >1000 |
| ebselen (EtOH) | 10.9 | >100 |
| remdesivir | 7.0 | >10 |

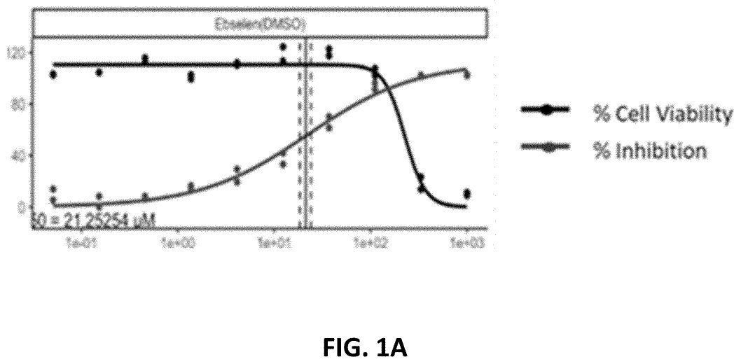

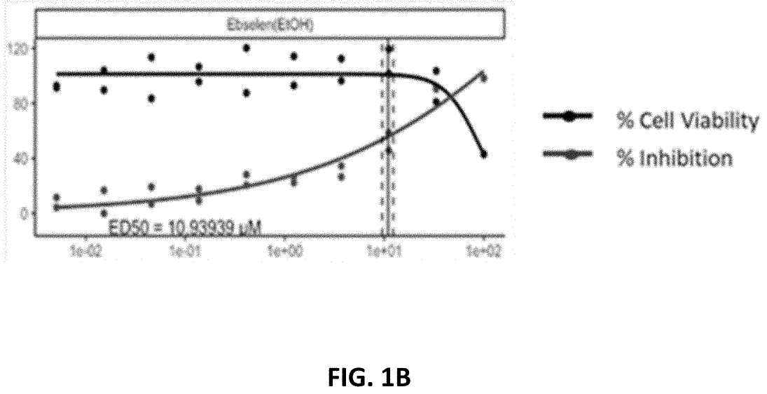

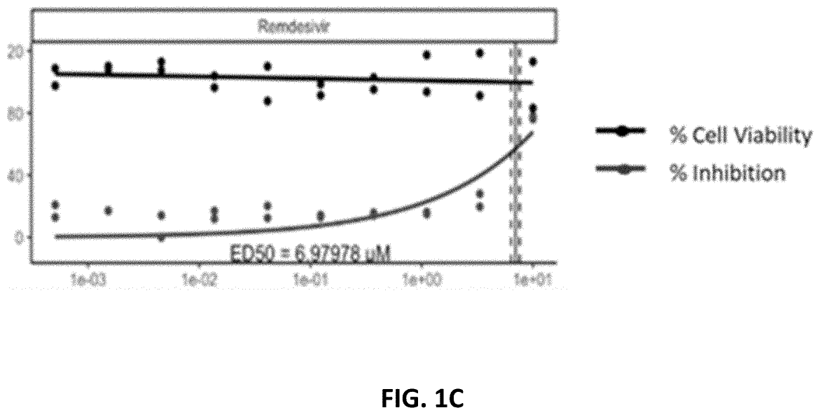

As seen in FIGS. 1A-C ebselen was able to inhibit viral load in the cell assay with EC₅₀ values of 21 μM (DMSO) or 11 μM (EtOH), respectively. While the cell viability was 100% at the EC₅₀, at slightly higher ranges cell viability was decreased. Remdesivir's EC₅₀ was 7 μM, and showed no effect on cell viability.

This is supported by Chen et al. (ACS Pharmacol. Transl. Sci. 2021, 4, 898-907 epub Apr. 9, 2021) which gives a possible mechanism for synergism between remdesivir and ebselen. They hypothesize that inhibition of SARS-CoV-2 proofreading activity by disulfiram/ebselen may allow remdesivir to escape nsp14 ExoN-mediated removal.

6.8. In Vivo Study of Ebselen in Hamster Model of Disease Transmission and Progression.

24 hours prior to nasal inoculation with SARS-CoV-2, Golden Syrian Hamsters were dosed with ebselen, saline and remdesivir at 15 mg/kg. Samples were taken from nares (PFU/swab) on days 1, 2 and 3, and from the nasal turbinates and cranial & caudal lung tissue (PFU/100 mg) on day 7.

6.8.1. Results

Table 2 shows that ebselen treatment resulted in lower counts in 6 of the 6 samples when compared to saline, and in 5 of 6 samples when compared to remdesivir. These results demonstrate ebselen's reduction of viral load in a hamster model of SARS-CoV-2 disease transmission and progression.

TABLE 2
Result of Viral Load Assay (plaque-forming units/PFU)

| | | | | PFU/100 mg | | |
|---|---|---|---|---|---|---|
| | PFU/swab (nares) | | | | Cranial | Caudal |
| Treatment | Day 1 | Day 2 | Day 3 | Turbinates | lung | lung |
| Saline | 2.7E+04 | 7.5E+03 | 5.5E+02 | 5.5E+07 | 4.9E+06 | 6.1E+06 |
| SPI-1005 | 1.0E+04 | 6.2E+03 | 1.7E+02 | 3.6E+07 | 2.3E+06 | 6.0E+06 |
| (ebselen) | | | | | | |
| Remdesivir | 1.1E+04 | 3.1E+03 | 3.8E+03 | 4.0E+02 | 4.4E+06 | 1.2E+07 |

6.9. Trial to Stop Ototoxicity in Cystic Fibrosis Patients with Acute Pulmonary Exacerbation Receiving IV Tobramycin

A trial was performed to assess the efficacy of SPI-1005 as a treatment for tobramycin-induced ototoxicity.

Thirty of 80 participants were enrolled starting in January 2019. They were Cystic fibrosis patients ages 18-70 that had acute pulmonary exacerbation or acute respiratory infections that required treatment with IV tobramycin 10 mg/kg/d for 10-21 days. They were randomized into four groups (1:1:1:1) (0, 200, 400, or 600 BID) and treated for 21 days. There were four follow-up visits for safety (adverse events, spirometry, labs), and two follow-up visits for efficacy (audiometric and vestibular). The primary endpoint was a reduction in cochleotoxic change, and the secondary endpoints was a reduction in tinnitus or vertigo severity.

6.9.1. Results

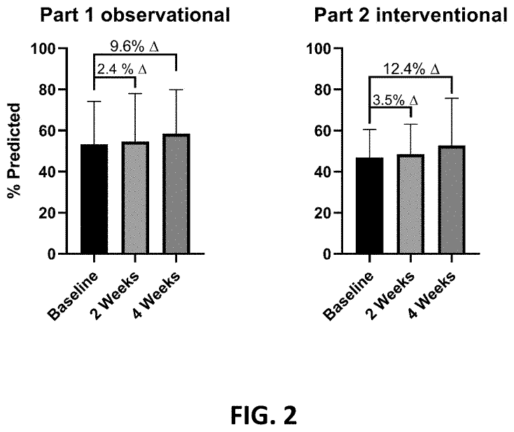

There was an increase or improvement in FEV1 over baseline at both 2 and 4 weeks as shown in table 3. This improvement in FEV1 was higher than what was documented in an observation study completed with 20 patients that were treated with IV tobramycin only (FIG. 2).

TABLE 3
FEV1 scores after intervention.

| | Mean (SD) | n |
|---|---|---|
| Baseline | 46.87 (13.68) | 23 |
| 2 Week | 48.53 (14.57) | 17 |
| 4 Week | 52.68 (22.99) | 19 |

There was no significant nephrotoxicity after IV tobramycin and SPI-1005 or placebo treatment. Table 4 shows the blood urea nitrogen (BUN), which are normally 5-22 mg/dl. Table 5 shows serum creatinine levels, which normally are 0.49-1.44 mg/dl.

TABLE 4
Blood Urea Nitrogen levels

| | Mean (SD) |
|---|---|
| Baseline | 12.81 (4.73) |
| Visit 2, on tobramycin and study treatment | 14.68 (4.80) |
| Visit 3, end of tobramycin treatment | 15.11 (5.56) |
| Visit 4, two weeks after end of tobramycin | 14.94 (6.66) |
| treatment | |
| Visit 5, four weeks after end of tobramycin | 13.45 (6.49) |
| treatment | |

TABLE 5
Serum Creatinine levels

| | Mean (SD) |
|---|---|
| Baseline | 0.77 (0.24) |
| Visit 2, on tobramycin and study treatment | 0.80 (0.22) |
| Visit 3, end of tobramycin treatment | 0.78 (0.20) |
| Visit 4, two weeks after end of tobramycin | 0.78 (0.19) |
| treatment | |
| Visit 5, four weeks after end of tobramycin | 0.81 (0.24) |
| treatment | |

Ototoxicity was higher in the observational than the interventional part of the study 4 weeks post-treatment as seen in table 6. The clinical chemistry results are shown in table 7.

TABLE 6
Results of intervention with SPI-1005 to prevent tobramycin-induced toxicity

| | | | | Conv + | DPO | | | | |
|---|---|---|---|---|---|---|---|---|---|
| | | Conventional | SRO | SRO | AE | WINT | TFI | TL | VSS |
| Part 1 | % Responders | 27% | 87% | 93% | 80% | 40% | 8% | 15% | 8% |
| Observation | % Responders | 27% | 53% | 60% | 40% | 33% | | | |
| | (Unilateral) | | | | | | | | |
| | % Responders | 0% | 33% | 33% | 40% | 7% | | | |
| | (Bilateral) | | | | | | | | |
| | No. of | 15 | 15 | 15 | 15 | 15 | 13 | 13 | 13 |
| | Subjects | | | | | | | | |
| | Assessed | | | | | | | | |
| Part 2 | % Responders | 10% | 58% | 57% | 80% | 26% | 14% | 0% | 5% |
| Intervention | % Responders | 5% | 37% | 29% | 40% | 26% | | | |
| (1:1:1:1) | (Unilateral) | | | | | | | | |
| | % Responders | 5% | 21% | 29% | 40% | 0% | | | |
| | (Bilateral) | | | | | | | | |
| | No. of | 21 | 19 | 21 | 20 | 19 | 22 | 22 | 22 |
| | Subjects | | | | | | | | |
| | Assessed | | | | | | | | |

TABLE 7
Clinical Chemistry Results
ITT Population

| | Screening/ | | | | | | | | | |
|---|---|---|---|---|---|---|---|---|---|---|
| | Baseline | | Visit 2 | | Visit 3 | | Visit 4 | | Visit 5 | |
| Sodium | 135.71 | (2.69) | 136.36 | (2.84) | 136.05 | (4.35) | 135.17 | (3.67) | 136.45 | (3.44) |
| Potassium | 4.33 | (0.44) | 4.22 | (0.44) | 4.25 | (0.35) | 4.16 | (0.31) | 4.25 | (0.65) |
| Chloride | 101.95 | (3.72) | 100.68 | (4.14) | 100.68 | (4.63) | 100.94 | (3.96) | 101.80 | (3.29) |
| Bicarbonate | 26.05 | (2.54) | 26.73 | (3.19) | 27.42 | (2.81) | 25.94 | (2.26) | 26.15 | (2.87) |
| BUN | 12.81 | (4.73) | 14.68 | (4.80) | 15.11 | (5.56) | 14.94 | (6.66) | 13.45 | (6.49) |
| Creatinine | 0.77 | (0.24) | 0.80 | (0.22) | 0.78 | (0.20) | 0.78 | (0.19) | 0.81 | (0.24) |
| Glucose | 101.24 | (57.19) | 120.95 | (74.45) | 102.53 | (46.88) | 132.56 | (98.20) | 119.05 | (76.70) |
| Total | 7.17 | (0.59) | 7.17 | (0.64) | 7.29 | (0.48) | 7.49 | (0.67) | 7.38 | (0.66) |
| Protein | | | | | | | | | | |
| Albumin | 3.80 | (0.54) | 3.87 | (0.45) | 4.05 | (0.36) | 4.08 | (0.54) | 3.98 | (0.56) |
| Calcium | 8.84 | (0.61) | 8.86 | (0.71) | 9.03 | (0.37) | 9.12 | (0.48) | 9.11 | (0.52) |
| Magnesium | 2.11 | (0.30) | 2.21 | (0.33) | 2.19 | (0.26) | 2.06 | (0.24) | 2.16 | (0.21) |
| Phosphorus | 3.62 | (0.67) | 3.61 | (0.76) | 3.68 | (0.84) | 3.84 | (0.66) | 3.72 | (0.82) |
| Uric Acid | 5.63 | (1.61) | 4.89 | (1.72) | 5 | (1.47) | 5.63 | (1.53) | 5.62 | (1.52) |
| Bilirubin | 0.46 | (0.30) | 0.41 | (0.27) | 0.45 | (0.15) | 0.49 | (0.29) | 0.46 | (0.28) |
| (Total) | | | | | | | | | | |
| ALT/SGPT | 17.05 | (10.46) | 31.41 | (26.06) | 35.63 | (25.94) | 20.44 | (13.43) | 22.45 | (15.66) |
| AST/SGOT | 16.86 | (4.95) | 26.45 | (17) | 27.58 | (15.02) | 18.22 | (8.61) | 26.60 | (34.47) |
| Alkaline | 97.71 | (33.46) | 93.41 | (43.41) | 90.16 | (45.79) | 104.50 | (38.18) | 101.70 | (39.55) |
| Phosphatase | | | | | | | | | | |
| GGT | 22 | (22.15) | 25 | (28.62) | 28.32 | (35.15) | 23.11 | (18.32) | 20.65 | (14.73) |
| LDH | 161.48 | (47.36) | 165.45 | (29.27) | 166.37 | (31.69) | 158.83 | (27.31) | 213.58 | (219.29) |
| Total | 126.43 | (51.42) | 139.68 | (33.60) | 151.89 | (46.05) | 134.78 | (58.30) | 137.60 | (51.08) |
| Cholesterol | | | | | | | | | | |
| Triglyceride | 78.33 | (30.64) | 114.82 | (61.32) | 114.84 | (44.50) | 96.06 | (48.34) | 89.95 | (34.57) |

This shows that SPI-1005 is well tolerated after 21 days of dosing during an acute respiratory infection requiring IV tobramycin and other standard of care. Additionally, no significant change in serum creatinine or blood urea nitrogen levels at 2 and 4 weeks follow-ups compared with baseline levels indicate a lack of nephrotoxicity.

In addition, these results show an improved relative FEV1% predicted at 2 and 4 weeks after IV tobramycin treatment that is greater than that in the completed Part 1 Observational study, potentially an added benefit of the anti-inflammatory MOA and Nrf2 activity of ebselen.

These results further show shows lower levels of ototoxic change at 4 weeks after IV tobramycin treatment than the Part 1 Observational study (Harruff et al., J Cyst Fibros. 2021 March; 20(2):288-294). Sentinel Pk showed no significant change in plasma tobramycin levels (lack of DDI). This reduction in tobramycin-induced ototoxicity is consistent with ebselen's otoprotective and neuroprotective properties observed in mice using auditory brainstem responses (Gu et al., J Cyst Fibros. 2021 March; 20(2):271-277). Ebselen-mediated improvements in tinnitus and hyperacusis have also been observed following amikacin-induced ototoxicity in mice (Longenecker et al., Front Neurosci. 2020 Sep. 18; 14:561185).

### EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

What is claimed is:

1. A method of treating a patient who has or is at risk for acute lung injury (ALI), acute respiratory distress syndrome (ARDS), ALI with concomitant pneumonia, or ARDS with concomitant pneumonia, comprising:
administering an effective amount of ebselen to a patient who has or is at risk for acute lung injury (ALI), acute respiratory distress syndrome (ARDS), ALI with concomitant pneumonia, or ARDS with concomitant pneumonia.

2. A method of treating a patient who has a confirmed or suspected viral lung infection, comprising:
administering a therapeutically effective amount of ebselen to a patient who is suffering from, or suspected of having, a viral lung infection.

3. A method of treating a patient who has or is at risk for cytokine release syndrome (CRS), comprising:
administering an effective amount of ebselen to a patient who has, or is at risk for, CRS.

4. The method of any one of claims 1-3, wherein the patient has a viral infection.

5. The method of claim 4, wherein the infection is by a virus selected from coronavirus, influenza virus, rhinovirus, respiratory syncytial virus, metapneumovirus, adenovirus, and boca virus.

6. The method of claim 5, wherein the virus is a coronavirus selected from coronavirus OC43, coronavirus 229E, coronavirus NL63, coronavirus HKU1, middle east respiratory syndrome beta coronavirus (MERS-CoV), severe acute respiratory syndrome beta coronavirus (SARS-CoV), and SARS-CoV-2 (COVID-19).

7. The method of claim 6, wherein the coronavirus is SARS-CoV-2 (COVID-19).

8. The method of any one of claims 1 to 7, wherein the patient has or is at risk for ALI.

9. The method of any one of claims 1 to 8, wherein the patient has or is at risk of ARDS.

10. The method of any one of claims 1 to 8, wherein the patient has or is at risk for ALI with concomitant pneumonia or ARDS with concomitant pneumonia.

11. The method of any one of claims 1 to 10, wherein the patient is asymptomatic or has mild symptoms of viral infection within 1 to 5 days prior to treatment.

12. The method of claim 11, wherein prior to treatment the patient is diagnosed as nCoV2 positive.

13. The method of claim 11 or 12, wherein the patient is naïve to treatment with an anti-viral agent (e.g., remdesivir, favilavir or galidesivir), hydroxychloroquine or azithromycin.

14. The method of any one of claims 11 to 13, wherein the ebselen is administered at 1 to 3 doses per day.

15. The method of claim 14, wherein the daily dose is 400 mg to 2000 mg.

16. The method of claim 15, wherein the ebselen is administered twice per day (BID).

17. The method of claim 16, wherein the twice per day (BID) dose is 400 mg.

18. The method of claim 16, wherein the twice per day (BID) dose is 600 mg.

19. The method of claim 16, wherein the twice per day (BID) dose is 800 mg.

20. The method of any one of claims 11-19, wherein ebselen is administered for seven days or more.

21. The method of any one of claims 11-20, further comprising assessing viral load in a sample of the patient.

22. The method of claim 21, wherein the viral load of the patient is assessed daily.

23. The method of claim 21 or 22, further comprising adjusting the daily dose of ebselen administered to the patient when the viral load is assessed as reduced by less than 1-log after 7 days of treatment.

24. The method of any one of claims 21-23, wherein the viral load of the patient is assessed as reduced by 1-log or more, 2-log or more, or 3-log or more within or after 7 days of treatment.

25. The method of any one of claims 1 to 10, wherein prior to treatment the patient has mild or moderate symptoms of viral infection.

26. The method of claim 25, wherein prior to treatment the patient is diagnosed as having SARS-CoV-2 (COVID-19).

27. The method of claim 25 or 26, wherein the patient is naïve to treatment with an anti-viral agent (e.g., remdesivir, favilavir or galidesivir).

28. The method of claim 25 or 26, wherein the patient is naïve to treatment with hydroxychloroquine or azithromycin.

29. The method of any one of claims 25 to 28, wherein the ebselen is administered at 1 to 3 doses per day.

30. The method of claim 29, wherein the daily dose is 400 mg to 2000 mg.

31. The method of claim 30, wherein the ebselen is administered twice per day (BID).

32. The method of claim 31, wherein the twice per day (BID) dose is 400 mg.

33. The method of claim 31, wherein the twice per day (BID) dose is 600 mg.

34. The method of claim 31, wherein the twice per day (BID) dose is 800 mg.

35. The method of any one of claims 25 to 34, wherein ebselen is administered for 14 to 21 days.

36. The method of any one of claims 25 to 34, wherein ebselen is administered for 21 days or more.

37. The method of any one of claims 25 to 36, wherein administering an effective amount of ebselen to the patient results in a reduction in the risk of respiratory morbidity and mortality.

38. The method of any one of claims 25 to 36, wherein administering an effective amount of ebselen to the patient results in a reduction in the patient's need for supplemental oxygen.

39. The method of any one of claims 25 to 36, wherein administering an effective amount of ebselen to the patient results in a reduction in the risk of assisted ventilation.

40. The method of any one of claims 25 to 36, wherein administering an effective amount of ebselen to the patient results in a reduction in the risk of ARDS.

41. The method of any one of claims 1 to 10, wherein the patient has moderate or severe symptoms of viral infection.

42. The method of claim 41, wherein prior to treatment the patient is diagnosed as having moderate or severe SARS-CoV-2 (COVID-19).

43. The method of claim 41 or 42, wherein the patient is naïve to treatment with an anti-viral agent (e.g., remdesivir, favilavir or galidesivir).

44. The method of claim 41 or 42, wherein the patient is naive to treatment with hydroxychloroquine or azithromycin.

45. The method of claim 41 or 42, wherein the patient is refractory to treatment with an anti-viral agent.

46. The method of claim 45, wherein the anti-viral agent is remdesivir, favilavir or galidesivir.

47. The method of claim 41 or 42, wherein the patient is refractory to treatment with hydroxychloroquine or azithromycin.

48. The method of any one of claims 41 to 47, wherein the ebselen is administered at 1 to 3 doses per day.

49. The method of claim 48, wherein the daily dose is 400 mg to 2000 mg.

50. The method of claim 49, wherein the ebselen is administered twice per day (BID).

51. The method of claim 50, wherein the twice per day (BID) dose is 400 mg.

52. The method of claim 50, wherein the twice per day (BID) dose is 600 mg.

53. The method of claim 50, wherein the twice per day (BID) dose is 800 mg.

54. The method of any one of claims 41-53, wherein ebselen is administered for 21 to 28 days.

55. The method of any one of claims 41-53, wherein ebselen is administered for 28 days or more.

56. The method of any one of claims 41-53, wherein prior to treatment the patient needs assisted ventilation, or is on a ventilator.

57. The method of any one of claims 41-53, wherein the ebselen is administered via nasogastric (ng) tube.

58. The method of any one of claims 41-57, wherein administering an effective amount of ebselen to the patient results in a reduction in the risk of respiratory failure.

59. The method of any one of claims 41-57, wherein administering an effective amount of ebselen to the patient results in eliminating the patient's need for assisted ventilation.

60. The method of any one of claims 41-57, further comprising adjusting the administered dose of ebselen to eliminate the patient's need for assisted ventilation.

61. The method of any one of claims 1 to 56, wherein the ebselen is administered orally.

62. The method of claim 61, wherein ebselen is formulated for oral administration in a solid dosage form.

63. The method of claim 57, wherein the solid dosage form is a capsule.

64. The method of any one of claims 1 to 51, wherein the ebselen is administered intravenously or by inhalation.

65. The method of any one of claims 1 to 64, wherein the patient is not hospitalized.

66. The method of any one of claims 1 to 64, wherein the patient is hospitalized.

67. The method of claim 66, wherein the patient is not on a ventilator.

68. The method of claim 67, wherein the administration of ebselen reduces or eliminates the patient's need for assisted ventilation.

69. The method of any one of claims 1 to 68, wherein the patient has a body temperature of greater than 37.5° C. prior to first administration of ebselen.

70. The method of claim 69, wherein the body temperature of the patient is measured at one or more sites selected from the group consisting of an oral cavity, a rectal cavity, axilla area, and tympanic membrane.

71. The method of claim 69 or 70 wherein the method reduces the body temperature of the patient below pre-treatment levels.

72. The method of any one of claims 1 to 71, wherein the patient has a pre-treatment C-creative protein (CRP) level greater than 2 mg/L.

73. The method of claim 72, wherein the patient has a pre-treatment CRP level greater than 5 mg/L, greater than 10 mg/L, greater than 20 mg/L, greater than 30 mg/L, or greater than 40 mg/L.

74. The method of any one of claims 1 to 73, wherein the method reduces the patient's serum CRP levels below pre-treatment levels.

75. The method of claim 74, wherein the post-treatment CRP level is no more than 45 mg/L, no more than 40 mg/L, no more than 35 mg/L, no more than 30 mg/L, no more than 20 mg/L, no more than 10 mg/L, no more than 5 mg/L, or no more than 1 mg/L.

76. The method of any one of claims 1-75, wherein the method reduces the CRP level by at least 10% (e.g., at least 20%, 30%, 40%, 50%, 60%, 70%, 80%, or 90%) as compared to pre-treatment levels.

77. The method of any one of claims 1 to 76, wherein the patient has a pre-treatment free IL-6 level in serum of at least 2 pg/ml.

78. The method of claim 77, wherein the patient has a pre-treatment free IL-6 level in serum of at least 2.5 pg/ml, 3 pg/ml, 4 pg/ml, 5 pg/ml, 10 pg/ml, 20 pg/ml, 30 pg/ml, 40 pg/ml, 50 pg/ml, 60 pg/ml, 70 pg/ml, 80 pg/ml, 90 pg/ml, 100 pg/ml, 150 pg/ml or 200 pg/ml.

79. The method of any one of claims 1 to 78, wherein the method reduces the patient's free IL-6 levels in serum below pre-treatment levels.

80. The method of claim 79, wherein the free IL-6 level in serum is decreased by at least 10% as compared to pre-treatment levels.

81. The method of claim 80, wherein the free IL-6 level in serum is decreased by at least 20%, 30%, 40%, 50%, 60%, 70%, 80%, or 90% as compared to pre-treatment levels.

82. The method of any one of claims 1 to 81, wherein the patient has a pre-treatment neutrophil-to-lymphocyte ratio (NLR) greater than 2.0.

83. The method of claim 82, wherein the patient has a pre-treatment NLR greater than 2.8, 2.9, 3.0, 3.1, 3.2, 3.3, 3.4, 3.5, 3.6, 3.7, 3.8, 3.9, or 4.0.

84. The method of claim 82, wherein the patient has a pre-treatment D-Dimer level that is elevated above baseline.

85. The method of claim 82, wherein the patient has a pre-treatment sepsis-induced coagulopathy (SIC) total score of 4 or more with total score of prothrombin time and coagulation exceeding 2.

86. The method of claim 83, wherein the patient has a post-treatment NLR less than 3.18.

87. The method of claim 86, wherein the administration of ebselen decreases the NLR by at least 10%, 20%, 30%, 40%, 50%, 60%, 70%, 80%, or 90% as compared to pre-treatment levels.

88. The method of any one of claims 1 to 87, wherein the patient has a pre-treatment respiration rate on ambient air of fewer than 12 breaths or more than 20 breaths per minute.

89. The method of claim 88, wherein the method improves the respiration rate of the patient.

90. The method of claim 89, wherein the patient has a post-treatment respiration rate between 12 to 20 breaths per minute.

91. The method of any one of claims 1 to 90, wherein the patient has a pre-treatment oxygen saturation level on ambient air of no more than 93%.

92. The method of any one of claims 1 to 90, wherein the patient has a pre-treatment oxygen saturation level on ambient air of no more than 85%, 80%, 75%, 70%, 65% or 60%.

93. The method of any one of claims 1 to 92, wherein the method improves the oxygen saturation level of the patient on ambient air by 10%, 20%, 30%, 40%, 50%, 60%, 70%, 80%, or 90% as compared to pre-treatment levels.

94. The method of any one of claims 1 to 93, wherein the method reduces the patient's need for supplemental oxygen.

95. The method of any one of claims 1 to 94, wherein the patient is older than 50, 51, 52, 53, 54, 55, 56, 57, 58, or 59 years of age.

96. The method of any one of claims 1 to 95, wherein the patient is older than 60 years of age.

97. The method of any one of claims 1 to 94, wherein the patient is younger than 60, 59, 58, 57, 56, 55, 54, 53, 52, 51, or 50 years of age.

98. The method of any one of claims 1 to 97, wherein the method further comprises administering an effective amount of at least one second therapeutic agent selected: an antiviral agent, antibacterial agent, an angiotensin receptor blocker (ARB), an IL-6 inhibitor, hydroxychloroquine, chloroquine, and COVID-19 immune serum or plasma.

99. The method of claim 98, wherein the at least one second therapeutic agent is an antiviral agent.

100. The method of claim 99, wherein the antiviral agent is favipiravir or galidesivir.

101. The method of claim 99, wherein the second therapeutic agent is remdesivir.

102. The method of claim 98, wherein the at least one second therapeutic agent is an antibacterial agent.

103. The method of claim 102, wherein the antibacterial agent is selected from the group consisting of azithromycin, tobramycin, aztreonam, ciprofloxacin, meropenem, cefepime, cetadizine, imipenem, piperacillin-tazobactam, amikacin, gentamicin and levofloxacin.

104. The method of claim 103, wherein the antibacterial agent is azithromycin.

105. The method of claim 98, wherein the at least one second therapeutic agent is an ARB.

106. The method of claim 105, wherein the ARB is losartan.

107. The method of claim 105, wherein the ARB is valsartan.

108. The method of claim 98, wherein the at least one second therapeutic agent is an IL-6 inhibitor.

109. The method of claim 108, wherein the IL-6 inhibitor is selected from the group consisting of: an anti-IL-6 receptor antibody or an antigen binding fragment thereof, an anti-IL-6 antibody or an antigen binding fragment thereof, and a JAK/STAT inhibitor.

110. The method of claim 109, wherein the IL-6 inhibitor is an anti-IL-6 receptor antibody, or antigen binding fragment thereof.

111. The method of claim 109, wherein the anti-IL-6 receptor antibody is tocilizumab or sarilumab.

112. The method of claim 109, wherein the IL-6 inhibitor is an anti-IL-6 antibody, or antigen binding fragment thereof.

113. The method of claim 112, wherein the anti-IL-6 antibody is selected from the group consisting of ziltivekimab, siltuximab, gerilimzumab, sirukumab, clazakizumab, olokizumab, VX30 (VOP-R003; Vaccinex), EB-007 (EBI-029; Eleven Bio), and FM101 (Femta Pharmaceuticals, Lonza).

114. The method of claim 109, wherein the IL-6 inhibitor is a JAK/STAT inhibitor.

115. The method of claim 114, wherein the JAK/STAT inhibitor is selected from the group consisting of ruxolotinib, tofacitinib, and baricitinib.

116. A pharmaceutical composition for treating acute lung injury (ALI), acute respiratory distress syndrome (ARDS), ALI with concomitant pneumonia, or ARDS with concomitant pneumonia, comprising:
ebselen;
an antiviral agent; and
a pharmaceutically acceptable excipient.

117. The composition of claim 116, wherein the anti-viral agent is selected from remdesivir, favilavir and galidesivir.

118. The composition of claim 117, wherein the anti-viral agent is remdesivir.
